(19) United States
(12) Patent Application Publication
(10) Pub. No.: US 2026/0328722 A1
(43) Pub. Date: Oct. 22, 2026

(54) CARBON NANOPARTICLE-BASED APTASENSOR FOR ELECTROCHEMICAL DETECTION OF GLIAL FIBRILLARY ACIDIC PROTEIN

(71) Applicant: University of Puerto Rico, San Juan, PR (US)

(72) Inventors: Yolimar Vazquez, San Juan, PR (US); Lisandro Cunci, San Juan, PR (US); Dasairy Santana, San Juan, PR (US)

(73) Assignee: University of Puerto Rico, San Juan, PR (US)

(21) Appl. No.: 19/553,281

(22) Filed: Feb. 28, 2026

Related U.S. Application Data
(60) Provisional application No. 63/764,574, filed on Feb. 28, 2025.

(51) Int. Cl. G01N 27/327; B82Y 15/00; G01N 27/36; G01N 33/68

(52) U.S. Cl. CPC G01N 27/3278; G01N 27/3276; G01N 27/36; G01N 33/68; B82Y 15/00; G01N 2333/47

(57) ABSTRACT

An electrochemical aptasensor for detection of glial fibrillary acidic protein includes a working electrode having a surface and a carbon-based ink disposed on the surface of the working electrode. The carbon-based ink includes chemically oxidized onion-like carbon nanoparticles having carboxylic acid groups. The carbon-based ink includes a single-stranded DNA aptamer specific for glial fibrillary acidic protein, wherein the single-stranded DNA aptamer is conjugated to the chemically oxidized onion-like carbon nanoparticles through the carboxylic acid groups via a crosslinker. The carbon-based ink includes a methylene blue redox reporter conjugated to the single-stranded DNA aptamer.

### CROSS-REFERENCE TO RELATED APPLICATIONS

This application claims the benefit of U.S. Provisional Patent Application No. 63/764,574, filed on Feb. 28, 2025, which is incorporated by reference herein in its entirety.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The invention relates in general to the field of electrochemical sensor, and more particularly, to electrochemical aptasensor for detection of glial fibrillary acidic protein.

### 2) Description of Related Art

Currently the state of the art includes numerous detection and treatments for Traumatic brain injuries.

Traumatic brain injuries represent a substantial health concern, contributing to morbidity, mortality, and long-term neurological impairment across diverse populations. The complexity inherent in existing diagnostic protocols has presented ongoing challenges in the identification and assessment of such injuries. Clinical evaluation and neuroimaging have remained standard approaches for decades; however, these methods may lack sensitivity for mild traumatic brain injuries and may not reliably differentiate injury severity at early stages. As a result, cases may remain undiagnosed or may be detected after neurological damage has progressed.

Blood and fluid-based biomarkers have emerged as complementary strategies for traumatic brain injury diagnosis. Among various biomarkers, glial fibrillary acidic protein has gained attention as an indicator of astrocytic injury, with elevated levels correlating with injury severity. Detection of glial fibrillary acidic protein through immunoassays such as enzyme-linked immunosorbent assay has demonstrated diagnostic utility. However, such methods may be time-consuming, may require centralized laboratory infrastructure, and may not be compatible with rapid, point-of-care applications. This creates interest in portable, sensitive biosensing platforms capable of detecting biomarkers in real time and in physiologically relevant matrices.

Nanomaterials have attracted attention in biosensing applications due to their large surface area, tunable surface chemistry, and electrical properties. Onion-like carbon nanoparticles provide conductivity and biocompatibility, while chemical oxidation can introduce carboxylic acid groups that enable bioconjugation. Aptamer-based electrochemical sensors, when combined with nanostructured conductive materials, offer potential for sensitivity, fast readout, and small device footprint. Incorporation of redox-active probes can enable real-time measurements and signal-off detection strategies suitable for biomarker quantification.

Accordingly, there exists interest in developing electrochemical biosensing platforms that integrate nanocarbon scaffolds, aptamer recognition, and electrochemical transduction for biomarker detection and monitoring, with potential utility in point-of-care diagnostics and decentralized monitoring technologies.

These and other objects, features, and advantages of the present invention will become more readily apparent from the attached drawings and the detailed description of the preferred embodiments, which follow.

### SUMMARY OF THE INVENTION

Bearing in mind the problems and deficiencies of the prior art, it is therefore an object of the present invention to provide an electrochemical aptasensor for detection of glial fibrillary acidic protein for the detection of traumatic brain injuries.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, an electrochemical aptasensor for detection of glial fibrillary acidic protein is provided. The electrochemical aptasensor includes a working electrode having a surface. The electrochemical aptasensor includes a carbon-based ink disposed on the surface of the working electrode. The carbon-based ink includes chemically oxidized onion-like carbon nanoparticles having carboxylic acid groups. The carbon-based ink includes a single-stranded DNA aptamer specific for glial fibrillary acidic protein, wherein the single-stranded DNA aptamer is conjugated to the chemically oxidized onion-like carbon nanoparticles through the carboxylic acid groups via a crosslinker. The carbon-based ink includes a methylene blue redox reporter conjugated to the single-stranded DNA aptamer.

According to other aspects of the present disclosure, the electrochemical aptasensor may include one or more of the following features. The crosslinker may be N-(3-Dimethylaminopropyl)-N-Ethylcarbodiimide. The single-stranded DNA aptamer may include a 5′-end having an amine group, wherein the amine group is conjugated to the carboxylic acid groups of the chemically oxidized onion-like carbon nanoparticles through the crosslinker. The single-stranded DNA aptamer may include a 3′-end having a thiol group, wherein the methylene blue redox reporter is conjugated to the thiol group via a thiol-maleimide Michael addition reaction. The methylene blue redox reporter may be methylene blue maleimide. The working electrode may be a glassy carbon electrode. The electrochemical aptasensor may be configured to exhibit a signal-off response upon binding of glial fibrillary acidic protein to the single-stranded DNA aptamer. The electrochemical aptasensor may be configured to detect glial fibrillary acidic protein in a concentration range from 0 pg/mL to 600 pg/mL. The electrochemical aptasensor may be configured to detect glial fibrillary acidic protein in a sweat sample.

According to another aspect of the present disclosure, a method of fabricating a carbon-based ink for an electrochemical aptasensor is provided. The method includes preparing a suspension of chemically oxidized onion-like carbon nanoparticles in deionized water. The method includes combining the suspension with a single-stranded DNA aptamer specific for glial fibrillary acidic protein and a crosslinker to form a first mixture. The method includes incubating the first mixture to conjugate the single-stranded DNA aptamer to the chemically oxidized onion-like carbon nanoparticles through carboxylic acid groups on the chemically oxidized onion-like carbon nanoparticles. The method includes adding a reducing agent to the first mixture to form a second mixture. The method includes adding a methylene blue redox reporter to the second mixture to conjugate the methylene blue redox reporter to the single-stranded DNA aptamer. The method includes purifying the second mixture to remove unbound reagents and obtain the carbon-based ink.

According to other aspects of the present disclosure, the method may include one or more of the following features. The suspension may have a concentration of 0.02 mg/mL of chemically oxidized onion-like carbon nanoparticles. The crosslinker may be N-(3-Dimethylaminopropyl)-N-Ethylcarbodiimide. The reducing agent may be Tris (2-Carboxyethyl) Phosphine Hydrochloride. The methylene blue redox reporter may be methylene blue maleimide. Incubating the first mixture may include incubating overnight at room temperature. Adding the reducing agent may include allowing the second mixture to stand for 2 hours. Purifying the second mixture may include dialysis using dialysis cassettes followed by centrifugation using centrifugal filter units. The method may further include depositing the carbon-based ink onto a surface of a working electrode via drop-casting.

According to another aspect of the present disclosure, a method of detecting glial fibrillary acidic protein in a sample is provided. The method includes providing an electrochemical aptasensor comprising a working electrode having a carbon-based ink disposed thereon. The carbon-based ink includes chemically oxidized onion-like carbon nanoparticles, a single-stranded DNA aptamer specific for glial fibrillary acidic protein conjugated to the chemically oxidized onion-like carbon nanoparticles, and a methylene blue redox reporter conjugated to the single-stranded DNA aptamer. The method includes contacting the electrochemical aptasensor with the sample. The method includes performing square wave voltammetry to measure an electrochemical signal from the methylene blue redox reporter. The method includes determining a concentration of glial fibrillary acidic protein in the sample based on a change in the electrochemical signal.

According to other aspects of the present disclosure, the method may include one or more of the following features. The sample may be a sweat sample. Performing square wave voltammetry may include scanning over a potential window from −0.5 V to 0.1 V versus Ag|AgCl at a frequency of 40 Hz. The electrochemical signal may exhibit a peak at approximately −0.2 V versus Ag|AgCl. Determining the concentration may include correlating a decrease in peak current of the electrochemical signal with an increase in glial fibrillary acidic protein concentration according to a signal-off detection mechanism. The method may further include using a three-electrode configuration comprising the working electrode, a platinum wire counter electrode, and an Ag|AgCl reference electrode.

The foregoing general description of the illustrative embodiments and the following detailed description thereof are merely exemplary aspects of the teachings of this disclosure and are not restrictive.

Still other objects and advantages of the invention will in part be obvious and will in part be apparent from the specification.

The above and other objects, which will be apparent to those skilled in the art, are achieved in the present invention which is directed to a n electrochemical aptasensor for detection of glial fibrillary acidic protein, comprising:
a. a working electrode having a surface; and
b. a carbon-based ink disposed on the surface of the working electrode, the carbon-based ink comprising:
c. chemically oxidized onion-like carbon nanoparticles having carboxylic acid groups;
d. a single-stranded DNA aptamer specific for glial fibrillary acidic protein, wherein the single-stranded DNA aptamer is conjugated to the chemically oxidized onion-like carbon nanoparticles through the carboxylic acid groups via a crosslinker; and
e. a methylene blue redox reporter conjugated to the single-stranded DNA aptamer.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 illustrates a flowchart depicting a process for surface modifications in glassy carbon electrodes, according to aspects of the present disclosure.

FIG. 2A depicts an X-ray diffraction spectrum of chemically oxidized onion-like carbon nanoparticles, according to an embodiment.

FIG. 2B depicts a Raman spectrum comparing modified onion-like carbon nanoparticles, diluted onion-like carbon nanoparticles, and bare highly ordered pyrolytic graphite, according to aspects of the present disclosure.

FIG. 3A depicts an atomic force microscopy image of chemically oxidized onion-like carbon nanoparticles before functionalization, according to an embodiment.

FIG. 3B depicts a three-dimensional atomic force microscopy surface map of chemically oxidized onion-like carbon nanoparticles before functionalization, according to aspects of the present disclosure.

FIG. 4A depicts a cyclic voltammetry graph showing electrochemical characterization of glassy carbon electrode surface modifications, according to an embodiment.

FIG. 4B depicts a graph showing electrochemical characterization of a glassy carbon electrode surface after methylene blue modification, according to aspects of the present disclosure.

FIG. 5A depicts a graph showing square wave voltammetry measurements at different glial fibrillary acidic protein concentrations in phosphate buffered saline, according to an embodiment.

FIG. 5B depicts a calibration curve showing the relationship between peak area and glial fibrillary acidic protein concentration in phosphate buffered saline, according to aspects of the present disclosure.

FIG. 6A depicts a graph showing square wave voltammetry measurements at different glial fibrillary acidic protein concentrations in phosphate buffered saline at pH 6.00, according to an embodiment.

FIG. 6B depicts a calibration curve for glial fibrillary acidic protein detection in phosphate buffered saline at pH 6.00, according to aspects of the present disclosure.

FIG. 7A depicts a graph showing square wave voltammetry measurements of glial fibrillary acidic protein concentrations in artificial sweat, according to an embodiment.

FIG. 7B depicts a calibration curve for glial fibrillary acidic protein detection in artificial sweat, according to aspects of the present disclosure.

FIG. 8A depicts a graph showing square wave voltammetry measurements of glial fibrillary acidic protein in real sweat samples, according to an embodiment.

FIG. 8B depicts a calibration curve for glial fibrillary acidic protein detection in a real sweat sample, according to aspects of the present disclosure.

FIG. 9 depicts a graph showing enzyme-linked immunosorbent assay results of glial fibrillary acidic protein concentrations obtained from sweat samples, according to an embodiment.

FIG. 10 depicts a graph showing electrochemical characterization of a glassy carbon electrode before and after modifications with chemically oxidized onion-like carbon nanoparticles, according to aspects of the present disclosure.

FIG. 11 depicts a graph showing electrochemical characterization via square wave voltammetry of in-solution modifications, according to an embodiment.

FIG. 12A depicts a graph showing a comparison of forward and reverse current via square wave voltammetry using a carbon-based ink at pH 7.00, according to aspects of the present disclosure.

FIG. 12B depicts a graph showing a comparison of forward and reverse current via square wave voltammetry using a carbon-based ink at pH 7.40, according to an embodiment.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

Prior to a discussion of the preferred embodiment of the invention, it should be understood that while the features and advantages of the invention are illustrated in terms of an electrochemical aptasensor platform for detection of glial fibrillary acidic protein (GFAP) the applicant realizes that the invention has numerous other applications and reserves those in advance.

The following description sets forth exemplary aspects of the present disclosure. It should be recognized, however, that such description is not intended as a limitation on the scope of the present disclosure. Rather, the description also encompasses combinations and modifications to those exemplary aspects described herein.

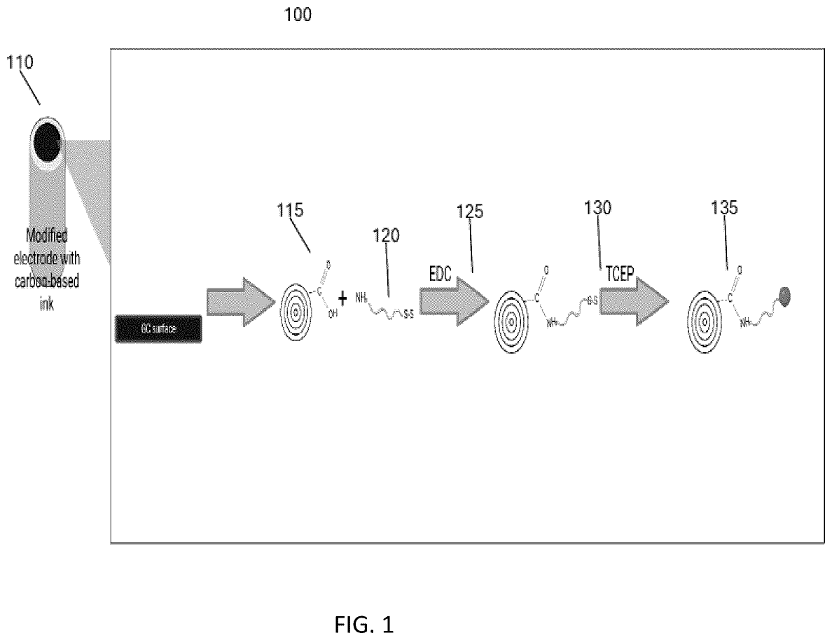

Referring to FIG. 1, the present disclosure relates to an electrochemical aptasensor platform for detection of glial fibrillary acidic protein (GFAP), which serves as a biomarker for traumatic brain injury. The electrochemical aptasensor integrates a nanostructured conductive scaffold, a molecular recognition element, and a redox reporter to achieve sensitive and selective electrochemical detection of GFAP in various sample matrices. FIG. 1 illustrates a process 100 for preparing a modified electrode with carbon-based ink 110 through sequential surface modifications of a glassy carbon electrode.

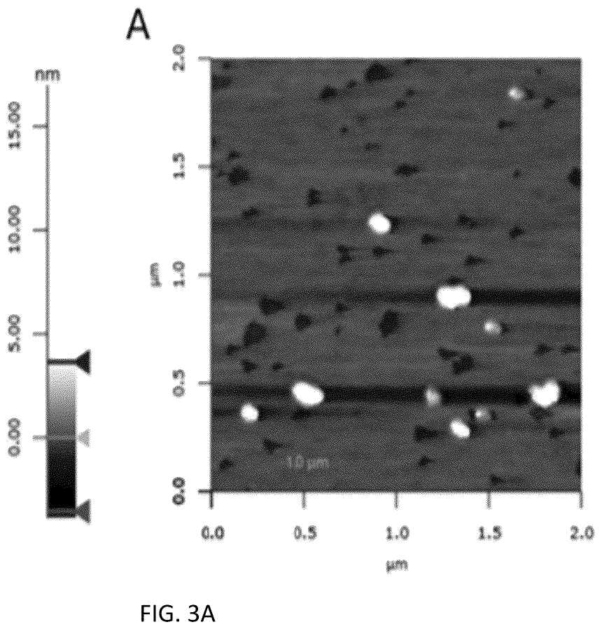

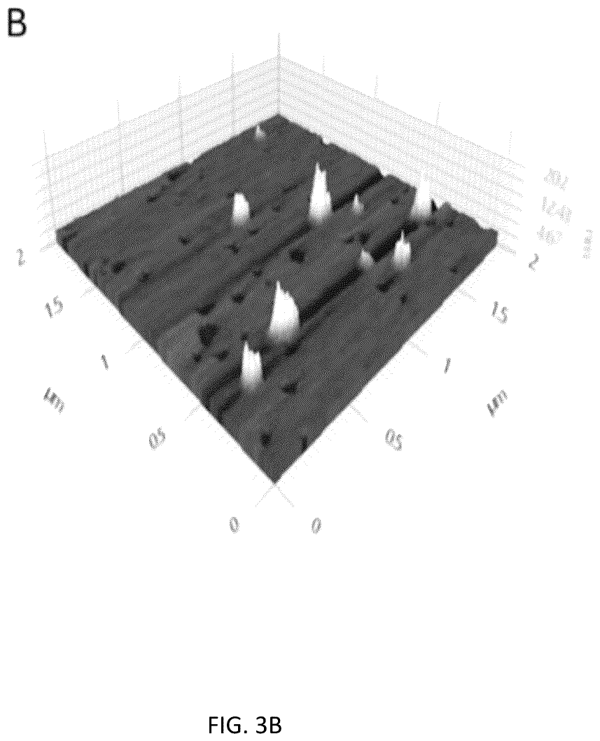

The process 100 employs chemically oxidized onion-like carbon nanoparticles 115 as the conductive nanoscaffold. The chemically oxidized onion-like carbon nanoparticles 115 provide a high surface area and tunable surface chemistry through abundant carboxylic acid groups that enable efficient bioconjugation. The chemically oxidized onion-like carbon nanoparticles 115 are prepared as a stock suspension by dispersing 1.0 mg of the nanoparticles in 50.0 mL of deionized water to yield a final concentration of 0.02 mg/mL. As shown in FIG. 3A and FIG. 3B, atomic force microscopy analysis reveals that the chemically oxidized onion-like carbon nanoparticles 115 have diameters up to approximately 9.61 nm.

With continued reference to FIG. 1, the process 100 incorporates a single-stranded DNA aptamer 120 that provides molecular recognition capability for GFAP. The single-stranded DNA aptamer 120 is a GFAP-specific aptamer designated as Gli_233. The single-stranded DNA aptamer 120 has a 5′-end that provides an amine group for attachment to the chemically oxidized onion-like carbon nanoparticles 115 via a crosslinker. The process 100 uses 2.95 mL of the chemically oxidized onion-like carbon nanoparticle suspension combined with 300 μL of the single-stranded DNA aptamer 120.

The process 100 utilizes N-(3-Dimethylaminopropyl)-N-Ethylcarbodiimide 125 (EDC) as a crosslinker to facilitate binding of the single-stranded DNA aptamer 120 to the chemically oxidized onion-like carbon nanoparticles 115. The process 100 uses 18.8 μL of a 5 mM solution of N-(3-Dimethylaminopropyl)-N-Ethylcarbodiimide 125. The mixture of chemically oxidized onion-like carbon nanoparticles 115, single-stranded DNA aptamer 120, and N-(3-Dimethylaminopropyl)-N-Ethylcarbodiimide 125 is incubated overnight at room temperature to allow the crosslinking reaction to proceed.

As further shown in FIG. 1, the process 100 employs Tris (2-Carboxyethyl) Phosphine Hydrochloride 130 (TCEP) as a reducing agent to prepare the single-stranded DNA aptamer 120 for subsequent conjugation with the redox probe. The process 100 uses 300 μL of a 5 mM solution of Tris (2-Carboxyethyl) Phosphine Hydrochloride 130. The reaction mixture is allowed to stand for 2 hours after addition of Tris (2-Carboxyethyl) Phosphine Hydrochloride 130.

The process 100 incorporates methylene blue maleimide 135 as the redox probe for electrochemical signal transduction. The process 100 uses 100 μL of a 1 mM solution of methylene blue maleimide 135. The single-stranded DNA aptamer 120 has a 3′-end that undergoes a thiol-maleimide Michael addition reaction to conjugate the methylene blue maleimide 135. The methylene blue maleimide 135 enables direct electrochemical signal transduction through a signal-off mechanism upon binding of GFAP to the single-stranded DNA aptamer 120.

Purification of the functionalized nanoconjugates is performed using Slide-A-Lyzer G3 Dialysis Cassettes with a molecular weight cutoff of 3,000 kDa. A sample volume of approximately 3.5 mL is transferred to the dialysis cassettes and completed with 4.0 mL of deionized water for a total volume of 7.5 mL inside the cassette membrane. The dialysis cassettes are placed in 2,000 mL beakers with deionized water and stirred at 150 rpm for 2 hours in the first cycle. For the second dialysis cycle, the deionized water is changed for fresh deionized water and the beakers are left stirring overnight to remove remnants of excess molecules.

The samples are transferred to Amicon centrifugal filter units with a molecular weight cutoff of 3,000 kDa in 50.0 mL Millipore tubes. The samples are centrifuged at 2,927×g (4,400 rpm) for 30 minutes during the purification process. Ultraviolet-visible spectroscopy is conducted to monitor the removal of excess methylene blue maleimide 135 during purification. The resulting carbon-based ink is stored in the dark to prevent photodegradation.

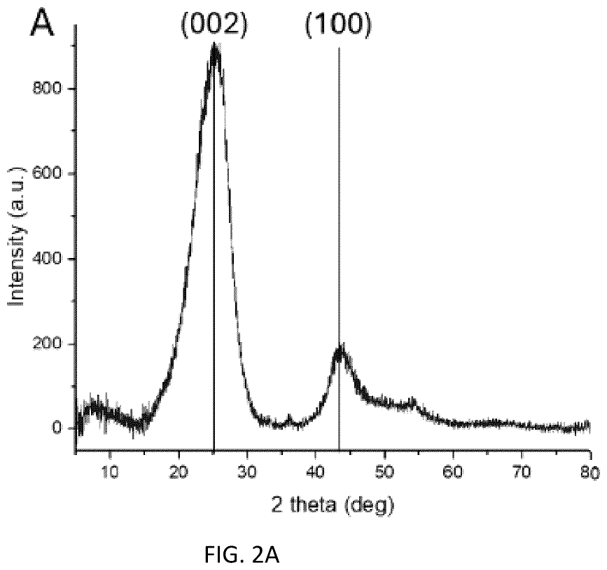

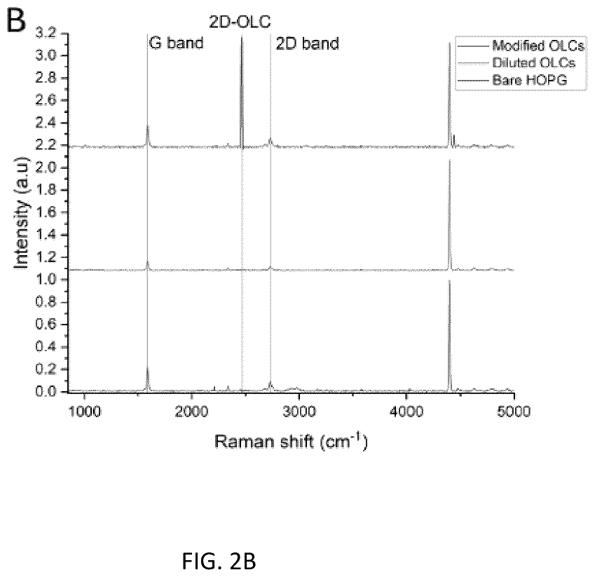

Referring to FIG. 2A, X-ray diffraction analysis of the chemically oxidized onion-like carbon nanoparticles 115 reveals characteristic diffraction peaks, including a (002) peak at approximately 26 degrees and a (100) peak at approximately 44 degrees, confirming the crystalline structure of the nanoparticles. FIG. 2B shows Raman spectroscopy analysis comparing modified onion-like carbon nanoparticles, diluted onion-like carbon nanoparticles, and bare highly ordered pyrolytic graphite substrate, demonstrating structural modification of the carbon lattice upon functionalization.

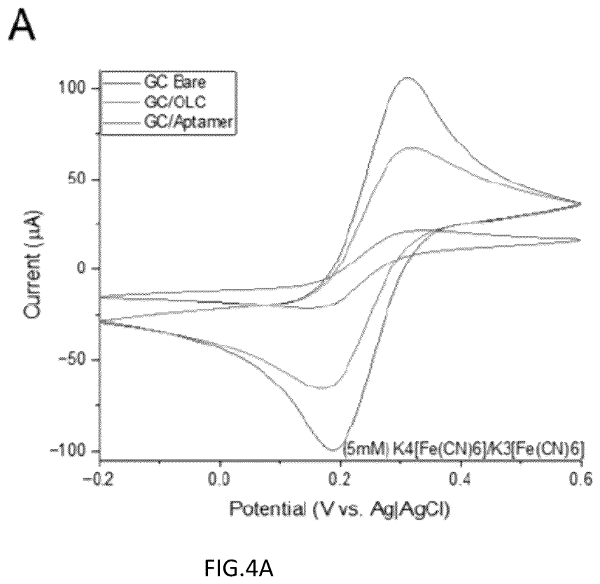

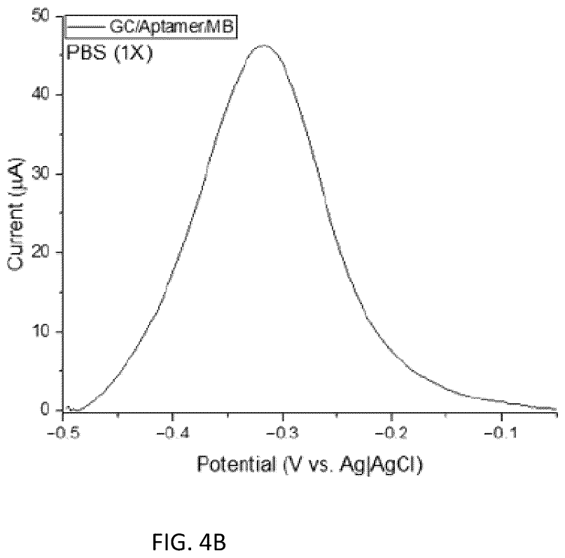

Referring to FIG. 4A, electrochemical characterization of the glassy carbon electrode surface modifications is performed using cyclic voltammetry in a solution of 5 mM potassium ferricyanide and potassium ferrocyanide in potassium chloride. The cyclic voltammograms demonstrate progressive changes in electron transfer resistance as the electrode surface is modified with the chemically oxidized onion-like carbon nanoparticles 115 and the single-stranded DNA aptamer 120. FIG. 4B shows the electrochemical response of the modified electrode after methylene blue conjugation, with the methylene blue maleimide 135 exhibiting an oxidation peak close to −0.2 V versus Ag|AgCl reference electrode.

The modified electrode with carbon-based ink 110 is deposited onto a glassy carbon electrode surface using a drop-casting method. The modified electrode with carbon-based ink 110 is configured for detection of glial fibrillary acidic protein in phosphate-buffered saline, artificial sweat, and real sweat samples. The modified electrode with carbon-based ink 110 operates using square wave voltammetry over a potential window from −0.5 to 0.1 V at a frequency of 40 Hz.

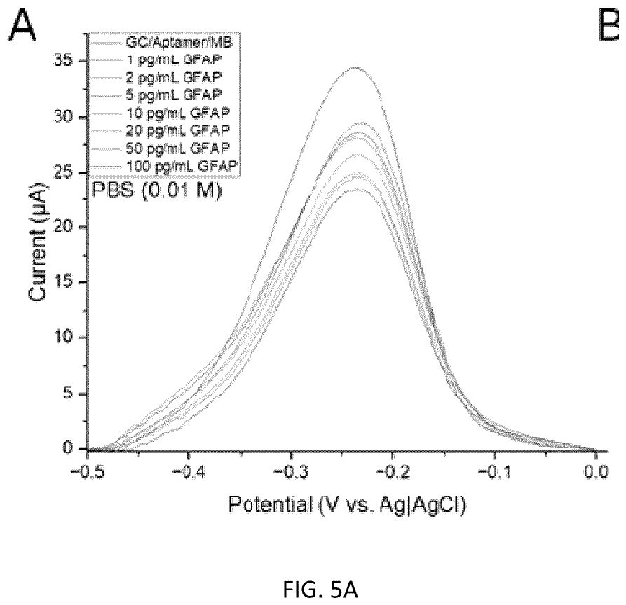

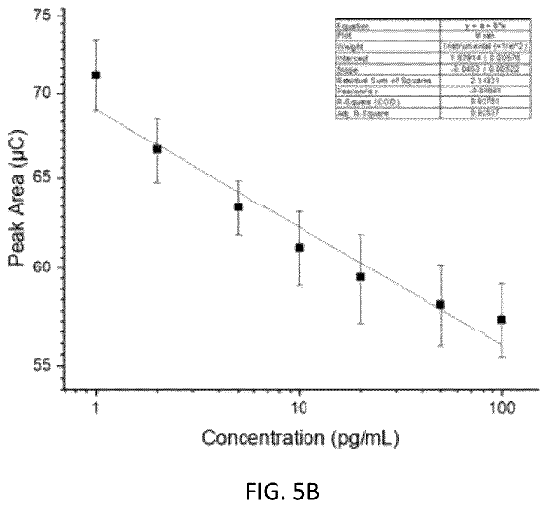

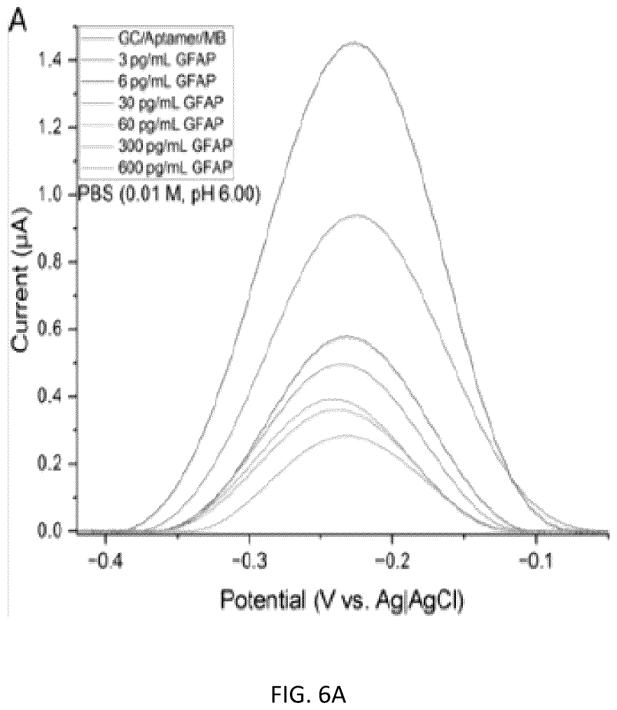

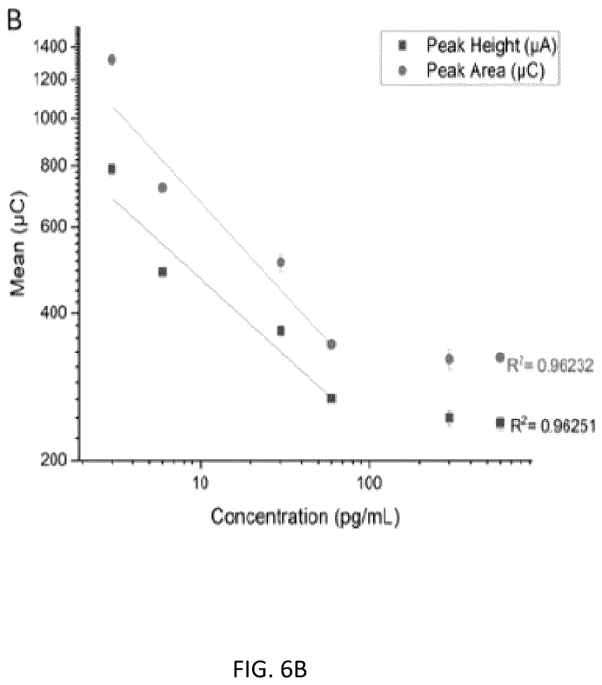

Referring to FIG. 5A and FIG. 5B, the modified electrode with carbon-based ink 110 exhibits a signal-off detection mechanism wherein the methylene blue peak current decreases with increasing GFAP concentration. FIG. 6A and FIG. 6B demonstrate that the modified electrode with carbon-based ink 110 achieves a linear calibration response with R²=0.96232 in PBS buffer at pH 6.00. The modified electrode with carbon-based ink 110 is capable of detecting GFAP concentrations ranging from 0 pg/mL up to 600 pg/mL.

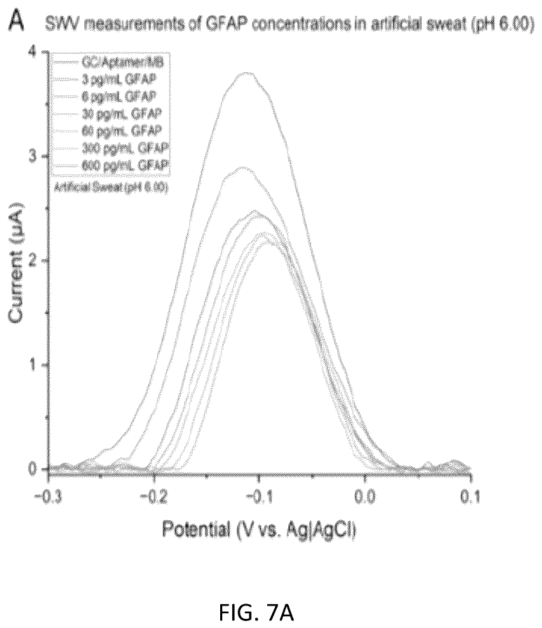

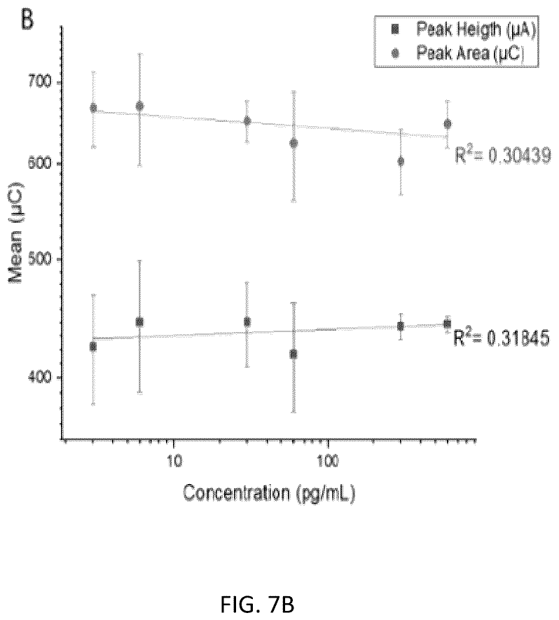

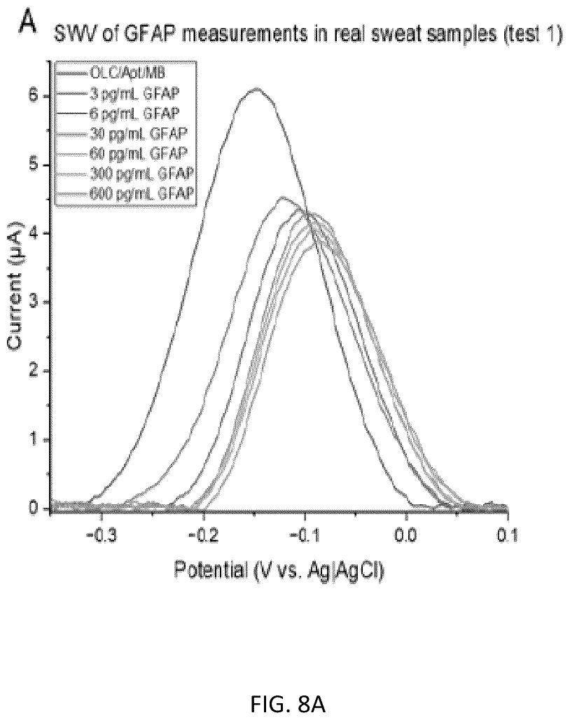

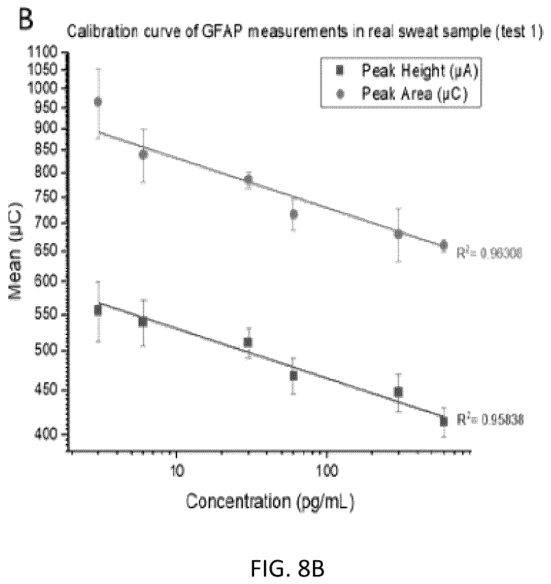

Referring to FIG. 7A and FIG. 7B, the modified electrode with carbon-based ink 110 is evaluated in artificial sweat prepared at pH 6.00 in accordance with ISO 105-E04 standards. FIG. 8A and FIG. 8B demonstrate that the modified electrode with carbon-based ink 110 achieves a linear calibration response with R²=0.96308 in real sweat samples, confirming the quantitative nature of the electrochemical response in physiologically relevant matrices.

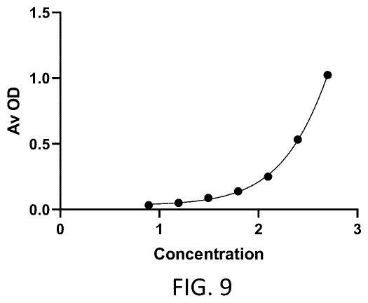

The modified electrode with carbon-based ink 110 uses a three-electrode configuration comprising a glassy carbon working electrode, a platinum wire counter electrode, and an Ag|AgCl reference electrode. The electrochemical cell has a maximum working volume of 250 μL. FIG. 9 shows ELISA validation results of GFAP concentrations obtained from sweat samples, demonstrating the analytical accuracy of the electrochemical aptasensor platform.

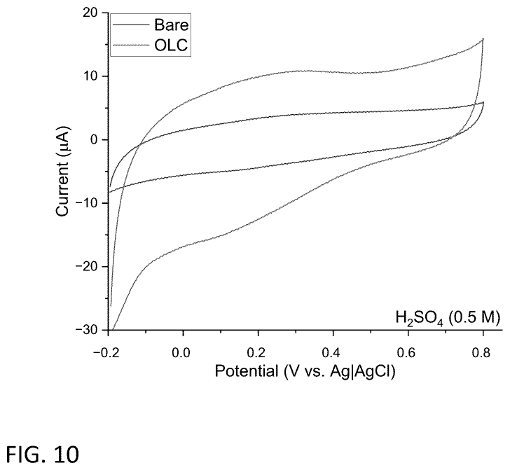

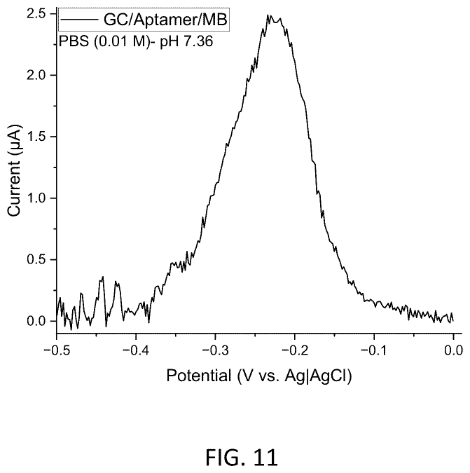

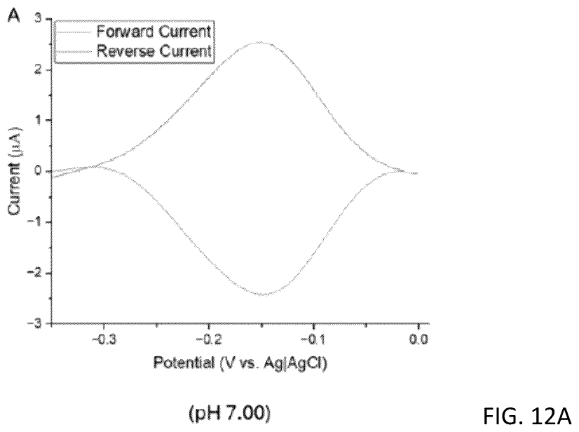

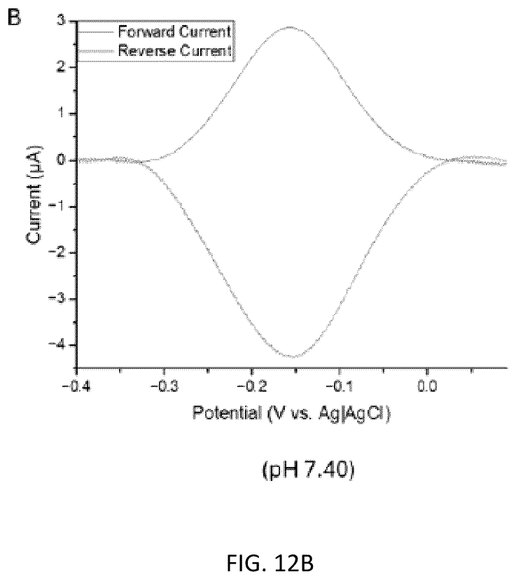

Referring to FIG. 10, electrochemical characterization of the glassy carbon electrode in sulfuric acid demonstrates the changes in electrochemical activity before and after modification with the chemically oxidized onion-like carbon nanoparticles 115. FIG. 11 shows background subtracted square wave voltammetry data of the in-solution modifications in phosphate-buffered saline. FIG. 12A and FIG. 12B illustrate comparisons of forward and reverse current via square wave voltammetry using the carbon-based ink at different pH values, demonstrating the reversible electrochemical behavior of the methylene blue maleimide 135 redox probe.

Referring to FIG. 1, the process 100 for surface modifications in glassy carbon electrodes proceeds through a sequential arrangement of components that interact to create a functionalized electrode capable of electrochemical detection of glial fibrillary acidic protein. The process 100 begins with the modified electrode with carbon-based ink 110, which serves as the foundation for the electrode preparation and subsequent surface modifications.

The chemically oxidized onion-like carbon nanoparticles 115 are deposited onto the electrode surface as the first modification step in the process 100. The chemically oxidized onion-like carbon nanoparticles 115 provide carboxylic acid groups that enable efficient bioconjugation with the single-stranded DNA aptamer 120. The carboxylic acid groups on the surface of the chemically oxidized onion-like carbon nanoparticles 115 serve as reactive sites for covalent attachment of subsequent components in the process 100.

With continued reference to FIG. 1, the single-stranded DNA aptamer 120 is introduced following deposition of the chemically oxidized onion-like carbon nanoparticles 115. The single-stranded DNA aptamer 120 is a GFAP-specific aptamer designated as Gli_233. The single-stranded DNA aptamer 120 has a 5′-end that provides an amine group for attachment to the chemically oxidized onion-like carbon nanoparticles 115 via the crosslinker. The amine group at the 5′-end of the single-stranded DNA aptamer 120 reacts with the carboxylic acid groups present on the chemically oxidized onion-like carbon nanoparticles 115 to form a covalent bond.

The N-(3-Dimethylaminopropyl)-N-Ethylcarbodiimide 125 functions as the crosslinker in the process 100 to facilitate binding of the single-stranded DNA aptamer 120 to the chemically oxidized onion-like carbon nanoparticles 115. The N-(3-Dimethylaminopropyl)-N-Ethylcarbodiimide 125 activates the carboxylic acid groups on the chemically oxidized onion-like carbon nanoparticles 115, enabling the crosslinking reaction with the amine group at the 5′-end of the single-stranded DNA aptamer 120.

As further shown in FIG. 1, the Tris (2-Carboxyethyl) Phosphine Hydrochloride 130 is added to the reaction mixture following the crosslinking step. The Tris (2-Carboxyethyl) Phosphine Hydrochloride 130 functions as a reducing agent that prepares the single-stranded DNA aptamer 120 for subsequent conjugation with the redox probe by reducing disulfide bonds to generate free thiol groups at the 3′-end of the single-stranded DNA aptamer 120.

The methylene blue maleimide 135 is introduced as the final component in the sequential process 100. The single-stranded DNA aptamer 120 has a 3′-end that undergoes a thiol-maleimide Michael addition reaction to conjugate the methylene blue maleimide 135. The thiol group at the 3′-end of the single-stranded DNA aptamer 120, generated by reduction with the Tris (2-Carboxyethyl) Phosphine Hydrochloride 130, reacts with the maleimide functional group of the methylene blue maleimide 135 to form a stable covalent linkage. The methylene blue maleimide 135 serves as the redox reporter that enables electrochemical signal transduction upon binding of glial fibrillary acidic protein to the single-stranded DNA aptamer 120.

Referring to FIG. 1, the preparation of the chemically oxidized onion-like carbon nanoparticles 115 as a stock suspension involves dispersing 1.0 mg of the chemically oxidized onion-like carbon nanoparticles 115 in 50.0 mL of deionized water. The dispersion yields a final concentration of 0.02 mg/mL of the chemically oxidized onion-like carbon nanoparticles 115 in the stock suspension. The stock suspension serves as the source material for subsequent combination with the single-stranded DNA aptamer 120 and the N-(3-Dimethylaminopropyl)-N-Ethylcarbodiimide 125.

With continued reference to FIG. 1, the process 100 combines 2.95 mL of the chemically oxidized onion-like carbon nanoparticle suspension with 300 μL of the single-stranded DNA aptamer 120. The single-stranded DNA aptamer 120 is a GFAP-specific aptamer designated as Gli_233. The single-stranded DNA aptamer 120 has a 5′-end that provides an amine group for attachment to the chemically oxidized onion-like carbon nanoparticles 115 via the N-(3-Dimethylaminopropyl)-N-Ethylcarbodiimide 125. The chemically oxidized onion-like carbon nanoparticles 115 provide carboxylic acid groups that enable efficient bioconjugation with the single-stranded DNA aptamer 120.

The process 100 uses 18.8 μL of a 5 mM solution of the N-(3-Dimethylaminopropyl)-N-Ethylcarbodiimide 125 as the crosslinker. The mixture of the chemically oxidized onion-like carbon nanoparticles 115, the single-stranded DNA aptamer 120, and the N-(3-Dimethylaminopropyl)-N-Ethylcarbodiimide 125 is incubated overnight at room temperature. The overnight incubation allows the crosslinking reaction between the carboxylic acid groups on the chemically oxidized onion-like carbon nanoparticles 115 and the amine group at the 5′-end of the single-stranded DNA aptamer 120 to proceed to completion.

As further shown in FIG. 1, the process 100 uses 300 μL of a 5 mM solution of the Tris (2-Carboxyethyl) Phosphine Hydrochloride 130 as the reducing agent. The reaction mixture is allowed to stand for 2 hours after addition of the Tris (2-Carboxyethyl) Phosphine Hydrochloride 130. The Tris (2-Carboxyethyl) Phosphine Hydrochloride 130 reduces disulfide bonds to generate free thiol groups at the 3′-end of the single-stranded DNA aptamer 120.

The process 100 uses 100 μL of a 1 mM solution of the methylene blue maleimide 135 as the redox probe. The single-stranded DNA aptamer 120 has a 3′-end that undergoes a thiol-maleimide Michael addition reaction to conjugate the methylene blue maleimide 135. The thiol groups generated by reduction with the Tris (2-Carboxyethyl) Phosphine Hydrochloride 130 react with the maleimide functional group of the methylene blue maleimide 135 to form a stable covalent linkage.

Purification of the functionalized nanoconjugates is performed using Slide-A-Lyzer G3 Dialysis Cassettes with a molecular weight cutoff of 3,000 kDa. A sample volume of approximately 3.5 mL is transferred to the dialysis cassettes and completed with 4.0 mL of deionized water for a total volume of 7.5 mL inside the cassette membrane. The dialysis cassettes are placed in 2,000 mL beakers with deionized water and stirred at 150 rpm for 2 hours in the first cycle. For the second dialysis cycle, the deionized water is changed for fresh deionized water and the beakers are left stirring overnight to remove remnants of excess molecules.

The samples are transferred to Amicon centrifugal filter units with a molecular weight cutoff of 3,000 kDa in 50.0 mL Millipore tubes. The samples are centrifuged at 2,927×g (4,400 rpm) for 30 minutes during the purification process. Ultraviolet-visible spectroscopy is conducted to monitor the removal of excess methylene blue maleimide 135 during purification. The resulting carbon-based ink is stored in the dark to prevent photodegradation.

The chemically oxidized onion-like carbon nanoparticles 115 have diameters up to approximately 9.61 nm as measured by atomic force microscopy. The modified electrode with carbon-based ink 110 is deposited onto a glassy carbon electrode surface using a drop-casting method. The modified electrode with carbon-based ink 110 is configured for detection of glial fibrillary acidic protein in phosphate-buffered saline, artificial sweat, and real sweat samples. The artificial sweat is prepared at pH 6.00 in accordance with ISO 105-E04 standards.

The modified electrode with carbon-based ink 110 operates using square wave voltammetry over a potential window from −0.5 to 0.1 V at a frequency of 40 Hz. The modified electrode with carbon-based ink 110 exhibits a signal-off detection mechanism wherein the methylene blue peak current decreases with increasing GFAP concentration. The methylene blue maleimide 135 exhibits an oxidation peak close to −0.2 V versus Ag|AgCl reference electrode.

The modified electrode with carbon-based ink 110 demonstrates a linear calibration response with R²=0.96232 in PBS buffer at pH 6.00. The modified electrode with carbon-based ink 110 demonstrates a linear calibration response with R²=0.96308 in real sweat samples. The modified electrode with carbon-based ink 110 is capable of detecting GFAP concentrations ranging from 0 pg/mL up to 600 pg/mL.

The modified electrode with carbon-based ink 110 uses a three-electrode configuration comprising a glassy carbon working electrode, a platinum wire counter electrode, and an Ag|AgCl reference electrode. The electrochemical cell has a maximum working volume of 250 μL.

Referring to FIG. 1, the process 100 includes an overnight incubation step at room temperature for conjugating the single-stranded DNA aptamer 120 to the chemically oxidized onion-like carbon nanoparticles 115. The mixture of the chemically oxidized onion-like carbon nanoparticles 115, the single-stranded DNA aptamer 120, and the N-(3-Dimethylaminopropyl)-N-Ethylcarbodiimide 125 is incubated overnight at room temperature to allow the crosslinking reaction to proceed. The overnight incubation period provides sufficient time for the N-(3-Dimethylaminopropyl)-N-Ethylcarbodiimide 125 to activate the carboxylic acid groups on the chemically oxidized onion-like carbon nanoparticles 115 and facilitate covalent bond formation with the amine group at the 5′-end of the single-stranded DNA aptamer 120.

With continued reference to FIG. 1, following the overnight incubation step, the process 100 includes addition of the Tris (2-Carboxyethyl) Phosphine Hydrochloride 130 as a reducing agent. The process 100 uses 300 μL of a 5 mM solution of the Tris (2-Carboxyethyl) Phosphine Hydrochloride 130. The reaction mixture is allowed to stand for 2 hours after addition of the Tris (2-Carboxyethyl) Phosphine Hydrochloride 130. The 2-hour standing period allows the Tris (2-Carboxyethyl) Phosphine Hydrochloride 130 to reduce disulfide bonds present at the 3′-end of the single-stranded DNA aptamer 120, thereby generating free thiol groups.

As further shown in FIG. 1, the free thiol groups generated by reduction with the Tris (2-Carboxyethyl) Phosphine Hydrochloride 130 are reactive toward the maleimide functional group of the methylene blue maleimide 135. The single-stranded DNA aptamer 120 has a 3′-end that undergoes a thiol-maleimide Michael addition reaction to conjugate the methylene blue maleimide 135. The reduction step with the Tris (2-Carboxyethyl) Phosphine Hydrochloride 130 prepares the single-stranded DNA aptamer 120 for subsequent conjugation with the methylene blue maleimide 135 by ensuring availability of free thiol groups for the Michael addition reaction.

Referring to FIG. 1, the process 100 uses 100 μL of a 1 mM solution of methylene blue maleimide 135 as the redox probe. The methylene blue maleimide 135 is introduced to the reaction mixture following the reduction step with the Tris (2-Carboxyethyl) Phosphine Hydrochloride 130. The solution is mixed thoroughly after introduction of the methylene blue maleimide 135 to ensure uniform distribution throughout the reaction mixture.

With continued reference to FIG. 1, the methylene blue maleimide 135 conjugates to the single-stranded DNA aptamer 120 through a thiol-maleimide Michael addition reaction at the 3′-end of the single-stranded DNA aptamer 120. The free thiol groups generated at the 3′-end of the single-stranded DNA aptamer 120 by the prior reduction step react with the maleimide functional group of the methylene blue maleimide 135. The thiol-maleimide Michael addition reaction forms a stable covalent thioether linkage between the single-stranded DNA aptamer 120 and the methylene blue maleimide 135.

The maleimide functional group of the methylene blue maleimide 135 is electrophilic and reacts selectively with the nucleophilic thiol groups present at the 3′-end of the single-stranded DNA aptamer 120. The Michael addition reaction proceeds under the mild conditions of the reaction mixture at room temperature. The resulting covalent linkage positions the methylene blue maleimide 135 at the 3′-end of the single-stranded DNA aptamer 120, enabling the methylene blue maleimide 135 to function as a redox reporter for electrochemical signal transduction.

Referring to FIG. 4B, the methylene blue maleimide 135 conjugated to the single-stranded DNA aptamer 120 exhibits an electrochemical response in phosphate-buffered saline. The methylene blue maleimide 135 displays an oxidation peak close to −0.2 V versus Ag|AgCl reference electrode. The electrochemical response confirms successful conjugation of the methylene blue maleimide 135 to the single-stranded DNA aptamer 120 through the thiol-maleimide Michael addition reaction.

As shown in FIG. 5B, the methylene blue maleimide 135 enables quantitative detection of glial fibrillary acidic protein through changes in peak area measured in microcoulombs. The calibration curve demonstrates an inversely proportional relationship between peak area and glial fibrillary acidic protein concentration, consistent with a signal-off detection mechanism. The methylene blue maleimide 135 positioned at the 3′-end of the single-stranded DNA aptamer 120 undergoes reduced electron transfer efficiency upon binding of glial fibrillary acidic protein to the single-stranded DNA aptamer 120.

Referring to FIG. 6B, the methylene blue maleimide 135 provides a linear calibration response with R²=0.96232 in PBS buffer at pH 6.00 when measuring peak area in microcoulombs. The calibration curve demonstrates that the methylene blue maleimide 135 conjugated through the thiol-maleimide Michael addition reaction at the 3′-end of the single-stranded DNA aptamer 120 enables reliable quantification of glial fibrillary acidic protein across a concentration range from 0 pg/mL up to 600 pg/mL. The negative slope of the calibration curve confirms the signal-off detection mechanism wherein increasing glial fibrillary acidic protein concentration results in decreasing electrochemical signal from the methylene blue maleimide 135.

Referring to FIG. 1, purification of the functionalized nanoconjugates resulting from the process 100 is performed using Slide-A-Lyzer G3 Dialysis Cassettes with a molecular weight cutoff of 3,000 kDa. The dialysis cassettes enable separation of the functionalized nanoconjugates from unbound reagents and excess molecules remaining in the reaction mixture following conjugation of the methylene blue maleimide to the single-stranded DNA aptamer attached to the chemically oxidized onion-like carbon nanoparticles.

With continued reference to FIG. 1, a sample volume of approximately 3.5 mL is transferred to the dialysis cassettes. The sample volume is completed with 4.0 mL of deionized water for a total volume of 7.5 mL inside the cassette membrane. The combined volume of sample and deionized water fills the dialysis cassette to an appropriate level for effective dialysis purification.

The dialysis cassettes are placed in 2,000 mL beakers with deionized water for the first dialysis cycle. The beakers containing the dialysis cassettes are stirred at 150 rpm for 2 hours during the first cycle. The stirring action promotes diffusion of unbound reagents and excess molecules across the dialysis membrane from the sample into the surrounding deionized water.

As further shown in FIG. 1, for the second dialysis cycle, the deionized water is changed for fresh deionized water. The replacement of the dialysis medium with fresh deionized water maintains a concentration gradient that drives continued removal of unbound reagents from the sample. The beakers are left stirring overnight during the second dialysis cycle to remove remnants of excess molecules from the functionalized nanoconjugates.

Referring to FIG. 1, following the dialysis purification steps, the samples are transferred to Amicon centrifugal filter units with a molecular weight cutoff of 3,000 kDa. The Amicon centrifugal filter units are placed in 50.0 mL Millipore tubes for the centrifugation process. The centrifugal filter units provide an additional purification step that concentrates the functionalized nanoconjugates while removing residual unbound molecules that passed through the dialysis membrane.

With continued reference to FIG. 1, the samples are centrifuged at 2,927×g, which corresponds to 4,400 rpm, for 30 minutes during the purification process. The centrifugal force drives the liquid phase through the filter membrane while retaining the functionalized nanoconjugates above the molecular weight cutoff threshold. The 30-minute centrifugation period provides sufficient time for effective separation and concentration of the functionalized nanoconjugates.

Ultraviolet-visible spectroscopy is conducted to monitor the removal of excess methylene blue maleimide during the purification process. The ultraviolet-visible spectroscopy measurements detect the characteristic absorption peaks of methylene blue in the filtrate, indicating the presence of unbound methylene blue maleimide that has been separated from the functionalized nanoconjugates. The spectroscopic monitoring confirms when the purification process has achieved adequate removal of excess methylene blue maleimide from the sample.

The resulting carbon-based ink is stored in the dark to prevent photodegradation. Methylene blue is susceptible to photodegradation when exposed to light, which degrades the redox activity of the methylene blue maleimide conjugated to the single-stranded DNA aptamer. Storage in the dark preserves the electrochemical properties of the carbon-based ink and maintains the functionality of the methylene blue maleimide as a redox reporter for subsequent electrochemical detection of glial fibrillary acidic protein.

Referring to FIG. 2A, the X-ray diffraction spectrum of chemically oxidized onion-like carbon nanoparticles before functionalization for carbon-based ink displays characteristic diffraction peaks that confirm the crystalline structure of the nanoparticles. The horizontal axis of the spectrum represents the diffraction angle (2 theta) measured in degrees, ranging from approximately 10 to 80 degrees. The vertical axis represents intensity measured in arbitrary units, ranging from 0 to approximately 900.

With continued reference to FIG. 2A, the spectrum displays a first diffraction peak labeled (002) at approximately 26 degrees with a maximum intensity of approximately 900 arbitrary units. The (002) peak represents the dominant crystallographic plane of the carbon nanoparticles and corresponds to the interlayer spacing of the graphitic carbon structure within the onion-like carbon nanoparticles. The position of the (002) peak at approximately 26 degrees is consistent with the expected diffraction angle for graphitic carbon materials.

As further shown in FIG. 2A, a second diffraction peak labeled (100) appears at approximately 44 degrees with a lower intensity of approximately 200 arbitrary units. The (100) peak corresponds to the in-plane structure of the graphitic carbon layers within the onion-like carbon nanoparticles. The presence of both the (002) and (100) diffraction peaks confirms that the chemically oxidized onion-like carbon nanoparticles retain the characteristic graphitic structure following the chemical oxidation process.

The broad nature of the diffraction peaks observed in FIG. 2A indicates the nanocrystalline character of the chemically oxidized onion-like carbon nanoparticles. The peak broadening results from the small crystallite size of the nanoparticles, as smaller crystallite dimensions produce broader diffraction peaks according to the Scherrer equation. The nanocrystalline character of the chemically oxidized onion-like carbon nanoparticles contributes to the high surface area that enables efficient bioconjugation with the single-stranded DNA aptamer.

The baseline of the spectrum shown in FIG. 2A exhibits minor fluctuations throughout the measured range, with the signal returning to near-zero intensity at higher diffraction angles beyond approximately 50 degrees. The absence of additional diffraction peaks at higher angles indicates that the chemically oxidized onion-like carbon nanoparticles do not contain detectable crystalline impurities or secondary phases that would interfere with the subsequent functionalization steps in the process for preparing the carbon-based ink.

Referring to FIG. 2B, the Raman spectrum compares three different samples: modified onion-like carbon nanoparticles, diluted onion-like carbon nanoparticles, and bare highly ordered pyrolytic graphite (HOPG). The x-axis of the spectrum represents the Raman shift measured in inverse centimeters, ranging from 1000 to 5000 cm⁻¹, while the y-axis represents intensity measured in arbitrary units, ranging from 0.0 to 3.2. The Raman spectroscopy analysis provides information about the vibrational modes and structural characteristics of the carbon materials at each stage of modification.

With continued reference to FIG. 2B, the bare HOPG sample exhibits sharp, well-defined peaks at characteristic positions with relatively low baseline intensity. The G band located at approximately 1600 cm⁻¹ corresponds to the in-plane stretching vibration of sp² carbon atoms in the graphitic lattice structure. The G band is a characteristic feature of graphitic carbon materials and indicates the presence of ordered graphitic domains within the sample. The 2D band at approximately 2750 cm⁻¹ represents a second-order Raman scattering process and provides information about the stacking order and number of graphene layers in the carbon material.

As further shown in FIG. 2B, the diluted onion-like carbon nanoparticle sample displays peaks at the same characteristic positions as the bare HOPG sample but with an elevated baseline intensity around 1.1 arbitrary units. The elevated baseline intensity in the diluted onion-like carbon nanoparticle sample results from the presence of the nanoparticles deposited on the HOPG substrate. The 2D-OLC peak at approximately 2450 cm⁻¹ is observed in the samples containing onion-like carbon nanoparticles and represents a characteristic vibrational mode associated with the oxidized onion-like carbon structure.

The modified onion-like carbon nanoparticle sample shown in FIG. 2B displays the highest baseline intensity at approximately 2.2 arbitrary units, with peaks at the same characteristic positions as the other samples. The increase in baseline intensity when comparing the modified onion-like carbon nanoparticles with the diluted onion-like carbon nanoparticles indicates structural modification upon functionalization with the aptamer and redox probe. The G band at approximately 1600 cm⁻¹ and the 2D band at approximately 2750 cm⁻¹ decrease in intensity following deposition of diluted onion-like carbon nanoparticles on the HOPG substrate; however, a slight increase in these bands is noted when comparing the chemically oxidized onion-like carbon nanoparticles with the aptamer and redox probe-functionalized chemically oxidized onion-like carbon nanoparticles.

The spectral features observed in FIG. 2B confirm that the fundamental carbon lattice structure is maintained throughout the functionalization process while demonstrating that structural modification occurs upon conjugation of the aptamer and redox probe to the chemically oxidized onion-like carbon nanoparticles. The presence of the G band, 2D-OLC peak, and 2D band in all samples containing onion-like carbon nanoparticles confirms the graphitic character of the nanoparticles. The changes in peak intensities and baseline levels between the diluted and modified samples provide evidence that the functionalization process alters the surface characteristics of the onion-like carbon nanoparticles while preserving the underlying graphitic structure that provides electrical conductivity for electrochemical signal transduction.

Referring to FIG. 3A, atomic force microscopy is employed to evaluate morphological characteristics and size distribution of the chemically oxidized onion-like carbon nanoparticles before aptamer and redox probe functionalization. FIG. 3A depicts a two-dimensional topographical scan of a sample surface with dimensions of approximately 2.0 micrometers by 2.0 micrometers. The surface displays a relatively flat substrate with visible horizontal striations or terraces characteristic of a highly ordered pyrolytic graphite substrate used for sample deposition.

With continued reference to FIG. 3A, scattered across the surface are multiple bright spots of varying sizes representing individual nanoparticles and small aggregates of the chemically oxidized onion-like carbon nanoparticles. A height scale bar on the left side of the image indicates elevation measurements ranging from approximately 0.00 nanometers to 15.00 nanometers, with the color gradient transitioning from dark brown at lower elevations to white at higher elevations. The nanoparticles appear as isolated bright circular or elliptical features with heights consistent with the expected size range for onion-like carbon nanoparticles. The darker triangular or irregular-shaped features distributed across the surface represent surface defects or contaminants on the substrate.

Referring to FIG. 3B, a three-dimensional atomic force microscopy surface map of the chemically oxidized onion-like carbon nanoparticles before aptamer and redox probe functionalization is depicted. The map displays a square scan area with dimensions of approximately 2 micrometers by 2 micrometers along the horizontal axes. The vertical axis indicates height measurements ranging from approximately 46 nanometers to 202 nanometers.

As further shown in FIG. 3B, the surface topography shows a relatively flat base layer with several distinct conical protrusions rising from the surface, representing individual or clustered nanoparticles. The protrusions appear as sharp peaks with heights extending above the surrounding surface, demonstrating the morphology and size distribution of the nanoparticles deposited on the substrate. The color gradient transitions from darker tones at lower elevations to lighter tones at the peak heights, providing visual contrast for the surface contour variations across the sample. The three-dimensional representation highlights the surface morphology and contour variations that characterize the chemically oxidized onion-like carbon nanoparticles prior to functionalization with the aptamer and redox probe.

The atomic force microscopy measurements reveal that the chemically oxidized onion-like carbon nanoparticles have diameters up to approximately 9.61 nm. The measured nanoparticle diameters are consistent with the expected size range for onion-like carbon nanoparticles. To minimize aggregation during sample preparation for atomic force microscopy analysis, onion-like carbon nanoparticle dispersions are diluted in isopropanol and deposited onto highly ordered pyrolytic graphite substrates to facilitate rapid drying. The particle size is expected to increase upon in-solution functionalization with the aptamer and methylene blue redox probe, reflecting successful conjugation of biomolecules to the nanoparticle surface.

Referring to FIG. 4A, electrochemical characterization of glassy carbon electrode surface modifications is performed using cyclic voltammetry in a solution of 5 mM potassium ferricyanide (K₃[Fe(CN)₆]) and potassium ferrocyanide (K₄[Fe(CN)₆]) in 0.1 M potassium chloride at a scan rate of 50 mV/s. FIG. 4A depicts a cyclic voltammetry graph that plots current in microamperes on the vertical axis against potential in volts versus Ag|AgCl on the horizontal axis. Three distinct curves are shown corresponding to different electrode states throughout the sequential modification process.

With continued reference to FIG. 4A, a black curve represents the bare glassy carbon electrode prior to any surface modifications. The bare electrode exhibits the highest peak currents among the three electrode states, with an oxidation peak reaching approximately 100 microamperes and a reduction peak reaching approximately negative 100 microamperes. The high peak currents observed for the bare electrode indicate efficient electron transfer between the electrode surface and the [Fe(CN)₆]³⁻/[Fe(CN)₆]⁴⁻ redox pair in the solution.

As further shown in FIG. 4A, a red curve represents the glassy carbon electrode after modification with chemically oxidized onion-like carbon nanoparticles. The electrode modified with chemically oxidized onion-like carbon nanoparticles shows intermediate peak currents compared to the bare electrode, with an oxidation peak of approximately 65 microamperes and a reduction peak of approximately negative 65 microamperes. The decrease in peak current magnitude following deposition of the chemically oxidized onion-like carbon nanoparticles indicates that the nanoparticle layer introduces electron transfer resistance at the electrode surface.

FIG. 4A further depicts a blue curve representing the glassy carbon electrode after aptamer immobilization on the chemically oxidized onion-like carbon nanoparticle-modified surface. The aptamer-modified electrode displays the lowest peak currents among the three electrode states, with an oxidation peak of approximately 25 microamperes and a reduction peak of approximately negative 25 microamperes. The further reduction in peak current magnitude following aptamer immobilization demonstrates additional surface blockage that impedes electron transfer between the electrode and the redox pair in solution.

The progressive decrease in peak current magnitude observed in FIG. 4A from the bare electrode to the chemically oxidized onion-like carbon nanoparticle-modified electrode to the aptamer-modified electrode confirms successful sequential modification of the electrode surface. The surface blockage generated by the chemically oxidized onion-like carbon nanoparticles and the aptamer increases electron transfer resistance between the solution and the electrode. The cyclic voltammetry characterization establishes that each modification step results in measurable changes in the electrochemical behavior of the electrode, providing confirmation that the chemically oxidized onion-like carbon nanoparticles and the aptamer are present on the electrode surface following the respective deposition and immobilization steps.

Referring to FIG. 4B, electrochemical characterization of the glassy carbon electrode surface after methylene blue modification is performed in 0.01 M phosphate-buffered saline (PBS) at pH 7.4. FIG. 4B depicts a graph showing the electrochemical response of the modified electrode, with current measured in microamperes on the vertical axis ranging from 0 to 50 microamperes, and potential measured in volts versus an Ag|AgCl reference electrode on the horizontal axis ranging from −0.5 to −0.1 volts.

With continued reference to FIG. 4B, a single curve labeled as GC/Aptamer/MB in PBS (1X) represents the electrochemical response of the glassy carbon electrode modified with the aptamer and methylene blue in 0.01 M phosphate-buffered saline at pH 7.4. The curve exhibits a characteristic peak with a maximum current of approximately 45 microamperes occurring at a potential near −0.3 volts versus Ag|AgCl. The methylene blue maleimide exhibits an oxidation peak close to −0.2 V versus Ag|AgCl reference electrode, confirming successful conjugation of the redox probe to the aptamer-functionalized electrode surface.

As further shown in FIG. 4B, the peak shape is asymmetric, with a steeper rise on the negative potential side and a more gradual decline on the positive potential side. The asymmetric peak shape is consistent with the expected electrochemical behavior of the methylene blue redox probe conjugated to the aptamer-functionalized electrode surface. The presence of the characteristic methylene blue oxidation peak demonstrates that the methylene blue maleimide retains electrochemical activity following conjugation to the single-stranded DNA aptamer through the thiol-maleimide Michael addition reaction at the 3′-end of the aptamer.

The electrochemical characterization shown in FIG. 4B confirms that the methylene blue maleimide functions as an effective redox reporter when conjugated to the aptamer on the modified electrode surface. The oxidation peak observed close to −0.2 V versus Ag|AgCl reference electrode provides the electrochemical signal that enables detection of glial fibrillary acidic protein through the signal-off mechanism, wherein binding of the target biomarker to the aptamer results in decreased electron transfer efficiency and corresponding reduction in the methylene blue peak current.

Referring to FIG. 5A, square wave voltammetry measurements are performed at different glial fibrillary acidic protein concentrations in 0.01 M PBS buffer at pH 7.40. FIG. 5A depicts a graph that plots current in microamperes on the vertical axis against potential in volts versus Ag|AgCl on the horizontal axis. The potential range extends from approximately −0.5 V to 0.0 V. Multiple overlapping curves are displayed, each representing a different glial fibrillary acidic protein concentration ranging from 0 pg/mL up to 100 pg/mL.

With continued reference to FIG. 5A, the curves include measurements at 0 pg/mL (labeled as GC/Aptamer/MB), 1 pg/mL, 2 pg/mL, 5 pg/mL, 10 pg/mL, 20 pg/mL, 50 pg/mL, and 100 pg/mL glial fibrillary acidic protein concentrations. Each curve exhibits a characteristic peak occurring at approximately −0.2 V, corresponding to the oxidation of the methylene blue redox probe conjugated to the aptamer. The peak current values range from approximately 22 to 35 microamperes across the concentration range tested.

As further shown in FIG. 5A, the modified electrode with carbon-based ink exhibits a signal-off detection mechanism wherein the methylene blue peak current decreases with increasing glial fibrillary acidic protein concentration. The graph demonstrates an inversely proportional relationship between glial fibrillary acidic protein concentration and peak current, where increasing concentrations of glial fibrillary acidic protein result in progressively lower peak currents. The highest peak current is observed at 0 pg/mL glial fibrillary acidic protein concentration, and the peak current decreases as the concentration increases toward 100 pg/mL.

Referring to FIG. 5B, a calibration curve depicts the relationship between peak area measured in microcoulombs and concentration of glial fibrillary acidic protein measured in picograms per milliliter in PBS buffer. The x-axis displays concentration on a logarithmic scale ranging from 1 to 100 pg/mL, while the y-axis displays peak area ranging from approximately 55 to 75 microcoulombs. Data points are represented as black squares with error bars indicating measurement variability at each concentration level.

With continued reference to FIG. 5B, a linear regression line with a negative slope extends through the data points, demonstrating the inversely proportional relationship between peak area and glial fibrillary acidic protein concentration. The negative slope of the calibration curve is consistent with the signal-off detection mechanism, wherein binding of glial fibrillary acidic protein to the aptamer reduces electron transfer efficiency from the methylene blue redox probe to the electrode surface. An inset table in the upper right portion of the graph displays statistical parameters including the equation form, intercept value, slope value, residual sum of squares, Pearson's r value, and R-Square coefficient of determination.

The signal-off detection mechanism observed in FIG. 5A and FIG. 5B results from conformational changes in the aptamer upon binding of glial fibrillary acidic protein. When glial fibrillary acidic protein binds to the aptamer, the aptamer undergoes a conformational change that increases the distance between the methylene blue redox probe and the electrode surface. The increased distance reduces the efficiency of electron transfer from the methylene blue to the electrode, resulting in decreased peak current and peak area measurements. The inversely proportional relationship between the electrochemical signal and glial fibrillary acidic protein concentration enables quantitative detection of the biomarker across the tested concentration range.

Referring to FIG. 6A, square wave voltammetry measurements are performed at different glial fibrillary acidic protein concentrations in 0.01 M PBS buffer at pH 6.00. FIG. 6A depicts a graph that plots current in microamperes on the vertical axis against potential in volts versus Ag|AgCl on the horizontal axis. The potential range extends from approximately −0.4 V to −0.1 V. Multiple curves are displayed corresponding to different glial fibrillary acidic protein concentrations, including a baseline measurement of the glassy carbon electrode modified with aptamer and methylene blue, and concentrations of 3 pg/mL, 6 pg/mL, 30 pg/mL, 60 pg/mL, 300 pg/mL, and 600 pg/mL glial fibrillary acidic protein.

With continued reference to FIG. 6A, each curve exhibits a peak centered near −0.2 V, which corresponds to the oxidation peak of the methylene blue redox probe. The curves demonstrate a progressive decrease in peak current with increasing glial fibrillary acidic protein concentration, consistent with the signal-off detection mechanism. The highest current response of approximately 1.4 microamperes is observed for the baseline measurement without glial fibrillary acidic protein, while the lowest current response of approximately 0.3 microamperes is observed at the highest glial fibrillary acidic protein concentration of 600 pg/mL.

Referring to FIG. 6B, a calibration curve depicts the relationship between concentration and mean signal response for glial fibrillary acidic protein detection in PBS buffer at pH 6.00. The x-axis represents concentration in pg/mL on a logarithmic scale ranging from approximately 1 to 1000 pg/mL. The y-axis represents the mean signal in μC, ranging from approximately 200 to 1400 μC. Two data series are plotted on the graph, with a first data series represented by black square markers corresponding to peak height measured in μA, and a second data series represented by red circular markers corresponding to peak area measured in μC.

As further shown in FIG. 6B, the peak area data series displays a linear regression fit line with an R² value of 0.96232, demonstrating a linear calibration response in PBS buffer at pH 6.00. The peak height data series displays a linear regression fit line with an R² value of 0.96251. Both data series demonstrate a negative correlation between concentration and signal response, consistent with the signal-off detection mechanism wherein binding of glial fibrillary acidic protein to the aptamer reduces electron transfer efficiency from the methylene blue redox probe.

The electrochemical aptasensor platform is capable of detecting glial fibrillary acidic protein concentrations ranging from 0 pg/mL up to 600 pg/mL in PBS buffer at pH 6.00. The extended concentration range from 0 to 600 pg/mL provides detection capability across physiologically relevant glial fibrillary acidic protein levels associated with traumatic brain injury. The linear calibration response with R²=0.96232 confirms the quantitative nature of the electrochemical response and demonstrates reliable correlation between the measured electrochemical signal and glial fibrillary acidic protein concentration across the tested range. Error bars visible on the data points in FIG. 6B indicate measurement variability at each concentration level, and the high R² values for both peak area and peak height measurements confirm reproducible sensor performance in PBS buffer at pH 6.00.

Referring to FIG. 7A, square wave voltammetry measurements are performed at different glial fibrillary acidic protein concentrations in artificial sweat at pH 6.00. The artificial sweat is prepared at pH 6.00 in accordance with ISO 105-E04 standards established by the International Organization for Standardization. FIG. 7A depicts a graph that plots current in microamperes on the vertical axis against potential in volts versus an Ag|AgCl reference electrode on the horizontal axis. The potential range extends from approximately −0.3 V to 0.1 V.

With continued reference to FIG. 7A, multiple curves are displayed representing different conditions including a baseline measurement with the glassy carbon electrode modified with aptamer and methylene blue, as well as measurements at various glial fibrillary acidic protein concentrations of 3 pg/mL, 6 pg/mL, 30 pg/mL, 60 pg/mL, 300 pg/mL, and 600 pg/mL in artificial sweat at pH 6.00. Each curve exhibits a peak centered near −0.1 V, corresponding to the oxidation of the methylene blue redox probe.

As further shown in FIG. 7A, the curves demonstrate a progressive decrease in peak current with increasing glial fibrillary acidic protein concentration, consistent with the signal-off detection mechanism where binding of the target biomarker to the aptamer reduces electron transfer efficiency. The highest peak current of approximately 3.5 microamperes corresponds to the baseline condition without glial fibrillary acidic protein, while the lowest peak currents of approximately 2.2 microamperes correspond to the higher glial fibrillary acidic protein concentrations.

Referring to FIG. 7B, a calibration curve depicts the relationship between concentration and mean signal response for glial fibrillary acidic protein detection in artificial sweat. The x-axis represents concentration in pg/mL on a logarithmic scale ranging from approximately 1 to 1000 pg/mL. The y-axis represents mean values in μC ranging from approximately 400 to 700.

With continued reference to FIG. 7B, two data series are plotted on the graph. A first data series, shown with circular markers and a corresponding trend line, represents peak area measurements in μC, with values ranging between approximately 600 and 680 μC across the concentration range, and displays an R² value of 0.30439. A second data series, shown with square markers and a corresponding trend line, represents peak height measurements in μA, with values ranging between approximately 420 and 460 μA across the concentration range, and displays an R² value of 0.31845.

As further shown in FIG. 7B, error bars are present on both data series indicating measurement variability at each concentration point. Both trend lines exhibit relatively flat slopes with slight negative correlations, indicating limited sensitivity of the sensor response in artificial sweat compared to PBS buffer. The low R² values for both peak area and peak height measurements indicate reduced linear correlation between glial fibrillary acidic protein concentration and electrochemical signal in the artificial sweat matrix compared to the PBS buffer matrix. The reduced correlation in artificial sweat results from the presence of additional ionic species and organic components in the artificial sweat formulation that affect the electrochemical environment at the electrode surface.

Referring to FIG. 8A, square wave voltammetry measurements are performed at different glial fibrillary acidic protein concentrations in real sweat samples. FIG. 8A depicts a graph that plots current in microamperes on the vertical axis against potential in volts versus a silver/silver chloride reference electrode on the horizontal axis. The potential range extends from approximately negative 0.3 volts to positive 0.1 volts, while the current range spans from 0 to approximately 6 microamperes.

With continued reference to FIG. 8A, multiple curves are displayed representing different concentrations of glial fibrillary acidic protein, including a baseline measurement with the onion-like carbon nanoparticle, aptamer, and methylene blue conjugate, as well as measurements at 3 picograms per milliliter, 6 picograms per milliliter, 30 picograms per milliliter, 60 picograms per milliliter, 300 picograms per milliliter, and 600 picograms per milliliter of glial fibrillary acidic protein. The baseline curve exhibits the highest peak current at approximately 6 microamperes, with the peak occurring near negative 0.15 volts.

As further shown in FIG. 8A, as the concentration of glial fibrillary acidic protein increases, the peak current progressively decreases, demonstrating a signal-off response mechanism. The curves at higher concentrations cluster together with peak currents ranging from approximately 3.5 to 4.5 microamperes, while maintaining similar peak positions near negative 0.1 volts. The concentration-dependent decrease in current is consistent with the expected behavior of an aptamer-based electrochemical sensor where target binding reduces electron transfer efficiency from the methylene blue redox reporter. The peak potential and peak shape remain largely unchanged across the concentration range, indicating that the redox chemistry of the methylene blue is preserved and that the observed decrease in signal results from reduced electron transfer efficiency rather than loss of reporter integrity.

Referring to FIG. 8B, a calibration curve depicts the relationship between glial fibrillary acidic protein concentration and mean signal response in a real sweat sample. The x-axis represents concentration in picograms per milliliter on a logarithmic scale ranging from approximately 1 to 100 pg/mL. The y-axis represents mean values in microcoulombs, ranging from approximately 400 to 1100 μC.

With continued reference to FIG. 8B, two data series are plotted on the graph. A first data series represented by square markers shows peak height measurements in microamperes, with values decreasing from approximately 550 μC at lower concentrations to approximately 420 μC at higher concentrations, following a linear regression fit with an R-squared value of 0.95838. A second data series represented by circular markers shows peak area measurements in microcoulombs, with values decreasing from approximately 960 μC at lower concentrations to approximately 660 μC at higher concentrations, following a linear regression fit with an R-squared value of 0.96308.

As further shown in FIG. 8B, error bars are displayed for each data point in both series, indicating measurement variability. Both trend lines demonstrate a negative slope, indicating an inverse relationship between glial fibrillary acidic protein concentration and the measured electrochemical signal. The negative correlation is consistent with the signal-off detection mechanism characteristic of the aptamer-based sensor, wherein binding of glial fibrillary acidic protein to the aptamer reduces electron transfer efficiency from the methylene blue redox probe to the electrode surface.

The electrochemical aptasensor platform configured for detection of glial fibrillary acidic protein in phosphate-buffered saline, artificial sweat, and real sweat samples demonstrates a linear calibration response with R²=0.96308 in real sweat samples. The high R² value confirms the quantitative nature of the electrochemical response in physiologically relevant matrices containing interferent molecules present in real sweat. The regression analysis demonstrates strong correlation between the measured electrochemical signal and glial fibrillary acidic protein concentration across the tested range from 0 pg/mL up to 600 pg/mL in real sweat samples.

Referring to FIG. 9, enzyme-linked immunosorbent assay (ELISA) results of glial fibrillary acidic protein concentrations obtained from sweat samples are depicted. FIG. 9 shows a graph that plots average optical density (Av OD) on the vertical axis against concentration on the horizontal axis. The vertical axis ranges from 0.0 to 1.5, while the horizontal axis ranges from 0 to 3. The data points form a sigmoidal curve characteristic of ELISA assays.

With continued reference to FIG. 9, the sigmoidal response demonstrates high sensitivity in the lower concentration range, where relatively small changes in analyte concentration produce measurable changes in optical density. At lower concentrations between approximately 0.5 and 1.5 on the horizontal axis, the average optical density values remain relatively low and stable. As concentration increases beyond approximately 2.0, the curve exhibits a steep increase in average optical density values, reflecting the transition region of the sigmoidal response where the assay demonstrates maximum sensitivity to concentration changes.

As further shown in FIG. 9, the curve approaches a plateau effect at higher concentrations, with the highest data point reaching approximately 1.0 average optical density at a concentration near 2.75. The plateau effect at higher concentrations results from saturation of the antibody binding sites in the ELISA assay, where additional analyte molecules do not produce proportional increases in optical density signal. The sigmoidal shape of the calibration curve is consistent with the binding kinetics of antibody-antigen interactions that underlie the ELISA detection mechanism.

The raw optical density values obtained from the ELISA measurements are averaged across replicates, corrected for background, and subsequently converted to concentrations using the standard calibration curve shown in FIG. 9. The calculated concentrations fall within the dynamic range of the assay, and the coefficient of variation values are within acceptable limits, confirming reproducibility of the measurements. Sample measurements yield concentrations of approximately 190.64 pg/mL and 567.95 pg/mL for different sweat samples, with both measurements above the assay blank and within the detection range.

The ELISA validation results confirm the analytical accuracy of the electrochemical aptasensor platform for glial fibrillary acidic protein detection in sweat samples. The concentrations determined by ELISA correlate with the electrochemical measurements obtained using the modified electrode with carbon-based ink, demonstrating that the aptasensor platform provides reliable quantification of glial fibrillary acidic protein in physiologically relevant matrices. The detection range of glial fibrillary acidic protein in sweat samples extends up to 540 pg/mL, and some samples fall outside this detection range, indicating the presence of elevated glial fibrillary acidic protein levels in certain sweat samples that exceed the upper limit of the ELISA assay dynamic range.

Referring to FIG. 10, electrochemical characterization of a glassy carbon electrode in 0.5 M sulfuric acid is performed before and after modifications with chemically oxidized onion-like carbon nanoparticles. FIG. 10 depicts a graph that plots current in microamperes on the vertical axis against potential in volts versus Ag|AgCl on the horizontal axis. The potential range extends from approximately −0.2 V to 0.8 V. Two cyclic voltammetry curves are shown, with a black line representing the bare electrode and a red line representing the electrode after modification with the chemically oxidized onion-like carbon nanoparticles.

With continued reference to FIG. 10, the bare electrode curve exhibits a relatively narrow hysteresis loop with current values ranging from approximately −10 microamperes to 10 microamperes. The narrow hysteresis loop observed for the bare glassy carbon electrode reflects the baseline electrochemical activity of the unmodified electrode surface in the sulfuric acid electrolyte. The cyclic voltammetry response of the bare electrode demonstrates characteristic oxidation and reduction processes occurring across the scanned potential window.

As further shown in FIG. 10, the modified electrode curve displays a substantially larger hysteresis loop with current values extending from approximately −30 microamperes to 10 microamperes. The larger hysteresis loop observed after modification with the chemically oxidized onion-like carbon nanoparticles indicates increased electrochemical activity following nanoparticle deposition onto the electrode surface. The expansion of the hysteresis loop results from the increased electroactive surface area provided by the chemically oxidized onion-like carbon nanoparticles deposited on the glassy carbon electrode.

The increase in current magnitude observed in the cathodic region of the cyclic voltammogram following nanoparticle modification demonstrates enhanced charge transfer at the electrode-electrolyte interface. The chemically oxidized onion-like carbon nanoparticles provide additional electroactive sites that participate in the electrochemical processes occurring during the potential sweep in sulfuric acid. The carboxylic acid groups introduced through chemical oxidation of the onion-like carbon nanoparticles contribute to the electrochemical response observed in the acidic electrolyte environment.

The electrochemical characterization in sulfuric acid confirms successful deposition of the chemically oxidized onion-like carbon nanoparticles onto the glassy carbon electrode surface. The measurable change in the cyclic voltammetry response between the bare electrode and the modified electrode provides evidence that the nanoparticle layer is present and electrochemically active on the electrode surface. The increased electrochemical activity demonstrated by the larger hysteresis loop establishes that the chemically oxidized onion-like carbon nanoparticles enhance the electrochemical properties of the glassy carbon electrode, which supports subsequent functionalization steps involving aptamer immobilization and redox probe conjugation.

Referring to FIG. 11, electrochemical characterization of background subtracted data via square wave voltammetry of the in-solution modifications is performed in 0.01 M phosphate-buffered saline at pH 7.36. FIG. 11 depicts a graph that displays current measured in microamperes on the vertical axis ranging from 0.0 to 2.5 microamperes, and potential measured in volts versus an Ag|AgCl reference electrode on the horizontal axis ranging from −0.5 to 0.0 volts. A single curve labeled as GC/Aptamer/MB represents the electrochemical response of a glassy carbon electrode modified with aptamer and methylene blue through the in-solution modification process.

With continued reference to FIG. 11, the curve shows a baseline current near zero at the more negative potentials around −0.5 volts, with some noise visible in this region. The current increases as the potential becomes less negative, reaching a peak current of approximately 2.4 microamperes at a potential near −0.22 volts. Following the peak, the current decreases as the potential approaches 0.0 volts, returning to near baseline levels with some residual noise.

As further shown in FIG. 11, the peak position near −0.2 volts is consistent with the expected oxidation potential of methylene blue. The characteristic methylene blue oxidation peak observed in the background subtracted square wave voltammetry data confirms successful conjugation of the redox probe to the aptamer-functionalized electrode surface through the in-solution modification process. The presence of the well-defined oxidation peak demonstrates that the methylene blue maleimide retains electrochemical activity following conjugation to the single-stranded DNA aptamer through the thiol-maleimide Michael addition reaction.

The background subtraction process removes contributions from non-faradaic currents and other background signals, isolating the faradaic response associated with the methylene blue redox probe. The resulting voltammogram provides a clear representation of the electrochemical behavior of the methylene blue conjugated to the aptamer on the electrode surface. The peak shape and position observed in FIG. 11 are consistent with the electrochemical response expected for methylene blue undergoing oxidation at the electrode-electrolyte interface.

The square wave voltammetry characterization in phosphate-buffered saline at pH 7.36 establishes the electrochemical functionality of the in-solution modified electrode prior to detection measurements. The peak current of approximately 2.4 microamperes provides a baseline signal level from which changes in current upon binding of glial fibrillary acidic protein to the aptamer are measured. The electrochemical response observed in FIG. 11 validates that the in-solution modification process produces a functional aptasensor with an electrochemically active methylene blue redox reporter positioned at the 3′-end of the aptamer for signal transduction during biomarker detection.

Referring to FIG. 12A, a comparison of forward and reverse current via square wave voltammetry using the carbon-based ink at pH 7.00 is depicted. FIG. 12A shows a graph that plots current in microamperes on the vertical axis against potential in volts versus Ag|AgCl on the horizontal axis. The potential range extends from approximately −0.3 V to 0.0 V. Two curves are displayed in the graph, with the forward current shown as a gray line and the reverse current shown as a red line.

With continued reference to FIG. 12A, the forward current curve exhibits a positive peak reaching approximately 2.5 microamperes at a potential near −0.15 V. The reverse current curve displays a corresponding negative peak reaching approximately −2.5 microamperes at a similar potential. Both curves cross through zero current at potentials near −0.3 V and 0.0 V, demonstrating the characteristic redox behavior of the methylene blue redox probe in the carbon-based ink formulation under the specified pH conditions.

The symmetrical positioning of the oxidation and reduction peaks around the same potential in FIG. 12A indicates reversible electrochemical behavior of the redox probe at pH 7.00. The forward current peak corresponds to the oxidation of methylene blue, while the reverse current peak corresponds to the reduction of the oxidized methylene blue species. The approximately equal magnitudes of the forward and reverse peak currents demonstrate that the oxidation and reduction processes occur with similar efficiency at pH 7.00.

Referring to FIG. 12B, a comparison of forward and reverse current via square wave voltammetry using the carbon-based ink at pH 7.40 is depicted. FIG. 12B shows a graph that plots current in microamperes on the vertical axis against potential in volts versus Ag|AgCl on the horizontal axis. The potential range extends from approximately −0.4 V to 0.0 V. A forward current trace is shown as a black line and a reverse current trace is shown as a red line.

With continued reference to FIG. 12B, the forward current trace exhibits a positive peak reaching approximately 2.7 microamperes at around −0.15 V. The reverse current trace exhibits a negative peak reaching approximately −4 microamperes at around −0.15 V. Both traces begin and end near zero current at the extremes of the potential window, with the forward current trace positioned above the zero line and the reverse current trace positioned below the zero line.

As further shown in FIG. 12B, the symmetrical positioning of the oxidation and reduction peaks around the same potential indicates reversible electrochemical behavior of the redox probe at pH 7.40. The peak positions remain consistent between pH 7.00 and pH 7.40, demonstrating that the methylene blue redox probe maintains electrochemical functionality across this pH range. The slight difference in peak current magnitudes between the forward and reverse traces at pH 7.40 reflects the influence of pH on the electron transfer kinetics of the methylene blue redox couple.

The comparison of the electrochemical responses at pH 7.00 and pH 7.40 demonstrates that the carbon-based ink containing the methylene blue-labeled aptamer conjugated to chemically oxidized onion-like carbon nanoparticles exhibits reversible redox behavior across physiologically relevant pH values. The preservation of the characteristic oxidation and reduction peaks at both pH values confirms that the methylene blue redox probe retains electrochemical activity and undergoes reversible electron transfer reactions regardless of the minor pH variations within this range. The reversible electrochemical behavior observed in FIG. 12A and FIG. 12B supports the use of the carbon-based ink for electrochemical detection of glial fibrillary acidic protein in biological matrices that exhibit pH values within this range.

The electrochemical aptasensor platform employs a three-electrode configuration for all electrochemical measurements. The three-electrode configuration comprises a glassy carbon working electrode, a platinum wire counter electrode, and an Ag|AgCl reference electrode filled with a 3 M NaCl solution. The glassy carbon working electrode serves as the substrate onto which the carbon-based ink is deposited and provides the surface at which the electrochemical detection of glial fibrillary acidic protein occurs. The platinum wire counter electrode completes the electrical circuit and allows current to flow through the electrochemical cell during measurements. The Ag|AgCl reference electrode provides a stable reference potential against which all potentials in the electrochemical measurements are expressed.

The electrochemical cell has a maximum working volume of 250 μL. The small working volume of the electrochemical cell enables measurements in limited sample volumes, which is advantageous for detection of biomarkers in biological fluids such as sweat where sample availability is constrained. The 250 μL working volume accommodates the three-electrode configuration while minimizing the quantity of sample required for each measurement.

The carbon-based ink is deposited onto the glassy carbon electrode surface using a drop-casting method. The drop-casting method involves dispensing a controlled volume of the carbon-based ink directly onto the surface of the glassy carbon working electrode. The deposited ink is allowed to dry on the electrode surface, forming a thin film of the functionalized nanoconjugates comprising the chemically oxidized onion-like carbon nanoparticles conjugated to the aptamer and methylene blue redox probe. The drop-casting deposition method provides a straightforward approach for applying the carbon-based ink to the electrode surface without requiring specialized equipment or complex fabrication procedures.

The electrochemical aptasensor platform operates using square wave voltammetry for detection of glial fibrillary acidic protein. Square wave voltammetry is performed over a potential window from −0.5 to 0.1 V versus the Ag|AgCl reference electrode. The potential window encompasses the oxidation potential of the methylene blue redox probe, which exhibits an oxidation peak close to −0.2 V versus Ag|AgCl. The square wave voltammetry measurements are conducted at a frequency of 40 Hz. The 40 Hz frequency provides a balance between measurement speed and signal resolution for detection of the methylene blue oxidation peak.

Square wave voltammetry offers advantages for electrochemical detection including enhanced sensitivity compared to other voltammetric techniques and effective discrimination against background currents. The square wave voltammetry technique applies a symmetrical square wave superimposed on a staircase potential waveform to the working electrode. The current is sampled at the end of each forward and reverse pulse, and the difference between the forward and reverse currents is recorded as the net current. The differential current measurement inherent to square wave voltammetry reduces contributions from capacitive charging currents and other non-faradaic processes, resulting in improved signal-to-noise ratio for detection of the faradaic current associated with the methylene blue redox probe.

The following is a list of figures and elements depicted in the drawings. FIG. 1 illustrates the process 100 for surface modifications in glassy carbon electrodes. FIG. 1 depicts the modified electrode with carbon-based ink 110, the chemically oxidized onion-like carbon nanoparticles 115, the single-stranded DNA aptamer 120, the N-(3-Dimethylaminopropyl)-N-Ethylcarbodiimide 125, the Tris (2-Carboxyethyl) Phosphine Hydrochloride 130, and the methylene blue maleimide 135. The process 100 proceeds through sequential steps wherein the chemically oxidized onion-like carbon nanoparticles 115 are prepared as a stock suspension by dispersing 1.0 mg of the nanoparticles in 50.0 mL of deionized water to yield a final concentration of 0.02 mg/mL. The process 100 uses 2.95 mL of the chemically oxidized onion-like carbon nanoparticle suspension combined with 300 μL of the single-stranded DNA aptamer 120. The process 100 uses 18.8 μL of a 5 mM solution of the N-(3-Dimethylaminopropyl)-N-Ethylcarbodiimide 125 as the crosslinker. The mixture of the chemically oxidized onion-like carbon nanoparticles 115, the single-stranded DNA aptamer 120, and the N-(3-Dimethylaminopropyl)-N-Ethylcarbodiimide 125 is incubated overnight at room temperature. The process 100 uses 300 μL of a 5 mM solution of the Tris (2-Carboxyethyl) Phosphine Hydrochloride 130 as the reducing agent. The reaction mixture is allowed to stand for 2 hours after addition of the Tris (2-Carboxyethyl) Phosphine Hydrochloride 130. The process 100 uses 100 μL of a 1 mM solution of the methylene blue maleimide 135 as the redox probe. The single-stranded DNA aptamer 120 is a GFAP-specific aptamer designated as Gli_233. The single-stranded DNA aptamer 120 has a 5′-end that provides an amine group for attachment to the chemically oxidized onion-like carbon nanoparticles 115 via the crosslinker. The single-stranded DNA aptamer 120 has a 3′-end that undergoes a thiol-maleimide Michael addition reaction to conjugate the methylene blue maleimide 135. The chemically oxidized onion-like carbon nanoparticles 115 provide carboxylic acid groups that enable efficient bioconjugation with the single-stranded DNA aptamer 120.

Purification of the functionalized nanoconjugates is performed using Slide-A-Lyzer G3 Dialysis Cassettes with a molecular weight cutoff of 3,000 kDa. A sample volume of approximately 3.5 mL is transferred to the dialysis cassettes and completed with 4.0 mL of deionized water for a total volume of 7.5 mL inside the cassette membrane. The dialysis cassettes are placed in 2,000 mL beakers with deionized water and stirred at 150 rpm for 2 hours in the first cycle. For the second dialysis cycle, the deionized water is changed for fresh deionized water and the beakers are left stirring overnight. The samples are transferred to Amicon centrifugal filter units with a molecular weight cutoff of 3,000 kDa in 50.0 mL Millipore tubes. The samples are centrifuged at 2,927×g (4,400 rpm) for 30 minutes during the purification process. Ultraviolet-visible spectroscopy is conducted to monitor the removal of excess methylene blue maleimide 135 during purification. The resulting carbon-based ink is stored in the dark to prevent photodegradation.

FIG. 2A depicts an X-ray diffraction (XRD) spectrum of the chemically oxidized onion-like carbon nanoparticles 115 before functionalization for carbon-based ink. The XRD spectrum displays characteristic diffraction peaks including a (002) peak at approximately 26 degrees and a (100) peak at approximately 44 degrees, confirming the crystalline structure of the nanoparticles.

FIG. 2B depicts a Raman spectrum comparing modified onion-like carbon nanoparticles, diluted onion-like carbon nanoparticles, and bare highly ordered pyrolytic graphite (HOPG). The Raman spectrum displays characteristic peaks including a G band at approximately 1600 cm⁻¹, a 2D-OLC peak at approximately 2450 cm⁻¹, and a 2D band at approximately 2750 cm⁻¹.

FIG. 3A depicts an atomic force microscopy (AFM) image of the chemically oxidized onion-like carbon nanoparticles 115 before aptamer and redox probe functionalization. The AFM image shows a two-dimensional topographical scan of a sample surface with dimensions of approximately 2.0 micrometers by 2.0 micrometers. The chemically oxidized onion-like carbon nanoparticles 115 have diameters up to approximately 9.61 nm as measured by atomic force microscopy.

FIG. 3B depicts a three-dimensional AFM surface map of the chemically oxidized onion-like carbon nanoparticles 115 before aptamer and redox probe functionalization. The 3D surface map displays a square scan area with dimensions of approximately 2 micrometers by 2 micrometers along the horizontal axes, with the vertical axis indicating height measurements.

FIG. 4A depicts cyclic voltammetry characterization of glassy carbon electrode surface modifications in a solution of 5 mM potassium ferricyanide and potassium ferrocyanide in potassium chloride at a scan rate of 50 mV/s. FIG. 4A shows three curves corresponding to the bare electrode, the electrode modified with the chemically oxidized onion-like carbon nanoparticles 115, and the electrode after aptamer immobilization.

FIG. 4B depicts electrochemical characterization of the glassy carbon electrode surface after methylene blue modification in 0.01 M phosphate-buffered saline at pH 7.4. The methylene blue maleimide 135 exhibits an oxidation peak close to −0.2 V versus Ag|AgCl reference electrode.

FIG. 5A depicts square wave voltammetry (SWV) measurements at different glial fibrillary acidic protein (GFAP) concentrations from 0 pg/mL up to 100 pg/mL in 0.01 M PBS buffer at pH 7.40. The modified electrode with carbon-based ink 110 exhibits a signal-off detection mechanism wherein the methylene blue peak current decreases with increasing GFAP concentration.

FIG. 5B depicts a calibration curve for GFAP in PBS buffer showing the relationship between peak area measured in microcoulombs and concentration of GFAP measured in picograms per milliliter.

FIG. 6A depicts square wave voltammetry measurements at different GFAP concentrations from 0 pg/mL up to 600 pg/mL in 0.01 M PBS buffer at pH 6.00. The modified electrode with carbon-based ink 110 is capable of detecting GFAP concentrations ranging from 0 pg/mL up to 600 pg/mL.

FIG. 6B depicts a calibration curve obtained for GFAP in PBS buffer at pH 6.00. The modified electrode with carbon-based ink 110 demonstrates a linear calibration response with R²=0.96232 in PBS buffer at pH 6.00.

FIG. 7A depicts square wave voltammetry measurements at different GFAP concentrations from 0 pg/mL up to 600 pg/mL in artificial sweat at pH 6.00. The artificial sweat is prepared at pH 6.00 in accordance with ISO 105-E04 standards.

FIG. 7B depicts a calibration curve obtained for GFAP in artificial sweat showing the relationship between concentration and mean signal response.

FIG. 8A depicts square wave voltammetry measurements at different GFAP concentrations from 0 pg/mL up to 600 pg/mL in a real sweat sample. The modified electrode with carbon-based ink 110 is configured for detection of glial fibrillary acidic protein in phosphate-buffered saline, artificial sweat, and real sweat samples.

FIG. 8B depicts a calibration curve for GFAP concentrations in real sweat. The modified electrode with carbon-based ink 110 demonstrates a linear calibration response with R²=0.96308 in real sweat samples.

FIG. 9 depicts ELISA results of GFAP concentrations obtained from sweat samples showing the relationship between average optical density and analyte concentration.

FIG. 10 depicts electrochemical characterization of a glassy carbon electrode in sulfuric acid (0.5 M) before and after modifications with the chemically oxidized onion-like carbon nanoparticles 115.

FIG. 11 depicts electrochemical characterization of background subtracted data via square wave voltammetry of the in-solution modifications in PBS (0.01 M) at pH 7.36.

FIG. 12A depicts a comparison of forward and reverse current via square wave voltammetry using the carbon-based ink at pH 7.00.

FIG. 12B depicts a comparison of forward and reverse current via square wave voltammetry using the carbon-based ink at pH 7.40.

The modified electrode with carbon-based ink 110 operates using square wave voltammetry over a potential window from −0.5 to 0.1 V at a frequency of 40 Hz. The modified electrode with carbon-based ink 110 is deposited onto a glassy carbon electrode surface using a drop-casting method. The modified electrode with carbon-based ink 110 uses a three-electrode configuration comprising a glassy carbon working electrode, a platinum wire counter electrode, and an Ag|AgCl reference electrode. The electrochemical cell has a maximum working volume of 250 μL.

A number of implementations have been described. Nevertheless, it will be understood that various modifications may be made without departing from the spirit and scope of the disclosure. Accordingly, other implementations are within the scope of the following claims.

The sensor of the current disclosure can be further described as an electrochemical aptasensor for detection of glial fibrillary acidic protein, comprising:
a. a working electrode having a surface; and
b. a carbon-based ink disposed on the surface of the working electrode, the carbon-based ink comprising:
   i. chemically oxidized onion-like carbon nanoparticles having carboxylic acid groups;
   ii. a single-stranded DNA aptamer specific for glial fibrillary acidic protein, wherein the single-stranded DNA aptamer is conjugated to the chemically oxidized onion-like carbon nanoparticles through the carboxylic acid groups via a crosslinker; and
   iii. a methylene blue redox reporter conjugated to the single-stranded DNA aptamer.
c. The electrochemical aptasensor of the current disclosure, wherein the crosslinker is N-(3-Dimethylaminopropyl)-N-Ethylcarbodiimide.
d. The electrochemical aptasensor of the current disclosure, wherein the single-stranded DNA aptamer comprises a 5′-end having an amine group, wherein the amine group is conjugated to the carboxylic acid groups of the chemically oxidized onion-like carbon nanoparticles through the crosslinker.

The electrochemical aptasensor of the current disclosure wherein the single-stranded DNA aptamer comprises a 3′-end having a thiol group, wherein the methylene blue redox reporter is conjugated to the thiol group via a thiol-maleimide Michael addition reaction.

The electrochemical aptasensor of the current disclosure, wherein the methylene blue redox reporter is methylene blue maleimide.

The electrochemical aptasensor of the current disclosure, wherein the working electrode is a glassy carbon electrode.

The electrochemical aptasensor of the current disclosure, wherein the electrochemical aptasensor is configured to exhibit a signal-off response upon binding of glial fibrillary acidic protein to the single-stranded DNA aptamer.

The electrochemical aptasensor of the current disclosure, wherein the electrochemical aptasensor is configured to detect glial fibrillary acidic protein in a concentration range from 0 pg/mL to 600 pg/mL.

The electrochemical aptasensor of the current disclosure, wherein the electrochemical aptasensor is configured to detect glial fibrillary acidic protein in a sweat sample.

The electrochemical aptasensor of the current disclosure, wherein the chemically oxidized onion-like carbon nanoparticles have diameters up to approximately 9.61 nm.

The electrochemical aptasensor of the current disclosure, wherein the single-stranded DNA aptamer is a GFAP-specific aptamer designated as Gli_233.

A method of fabricating a carbon-based ink the current disclosure for an electrochemical aptasensor, comprising:
a. preparing a suspension of chemically oxidized onion-like carbon nanoparticles in deionized water;
b. combining the suspension with a single-stranded DNA aptamer specific for glial fibrillary acidic protein and a crosslinker to form a first mixture;
c. incubating the first mixture to conjugate the single-stranded DNA aptamer to the chemically oxidized onion-like carbon nanoparticles through carboxylic acid groups on the chemically oxidized onion-like carbon nanoparticles;
d. adding a reducing agent to the first mixture to form a second mixture;
e. adding a methylene blue redox reporter to the second mixture to conjugate the methylene blue redox reporter to the single-stranded DNA aptamer; and
f. purifying the second mixture to remove unbound reagents and obtain the carbon-based ink.

The method of the current disclosure, wherein the suspension has a concentration of 0.02 mg/mL of chemically oxidized onion-like carbon nanoparticles.

The method of the current disclosure, wherein:
a. the crosslinker is N-(3-Dimethylaminopropyl)-N-Ethylcarbodiimide;
b. the reducing agent is Tris (2-Carboxyethyl) Phosphine Hydrochloride; and
c. the methylene blue redox reporter is methylene blue maleimide.

The method of the current disclosure, wherein incubating the first mixture comprises incubating overnight at room temperature, and wherein adding the reducing agent comprises allowing the second mixture to stand for 2 hours.

The method of the current disclosure, wherein purifying the second mixture comprises dialysis using dialysis cassettes followed by centrifugation using centrifugal filter units.

A method of detecting glial fibrillary acidic protein in a sample, comprising: providing an electrochemical aptasensor comprising a working electrode having a carbon-based ink disposed thereon, the carbon-based ink comprising:
a. chemically oxidized onion-like carbon nanoparticles;
b. a single-stranded DNA aptamer specific for glial fibrillary acidic protein conjugated to the chemically oxidized onion-like carbon nanoparticles; and
c. a methylene blue redox reporter conjugated to the single-stranded DNA aptamer;
d. contacting the electrochemical aptasensor with the sample;
e. performing square wave voltammetry to measure an electrochemical signal from the methylene blue redox reporter; and
f. determining a concentration of glial fibrillary acidic protein in the sample based on a change in the electrochemical signal.

The method of the current disclosure, wherein the sample is a sweat sample.

The method of the current disclosure, wherein performing square wave voltammetry comprises scanning over a potential window from −0.5 V to 0.1 V versus Ag|AgCl at a frequency of 40 Hz, and wherein the electrochemical signal exhibits a peak at approximately −0.2 V versus Ag|AgCl.

The method of the current disclosure, wherein determining the concentration comprises correlating a decrease in peak current of the electrochemical signal with an increase in glial fibrillary acidic protein concentration according to a signal-off detection mechanism.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments; it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

We claim:

1. An electrochemical aptasensor for detection of glial fibrillary acidic protein, comprising:
a working electrode having a surface; and
a carbon-based ink disposed on the surface of the working electrode, the carbon-based ink comprising:
chemically oxidized onion-like carbon nanoparticles having carboxylic acid groups;
a single-stranded DNA aptamer specific for glial fibrillary acidic protein, wherein the single-stranded DNA aptamer is conjugated to the chemically oxidized onion-like carbon nanoparticles through the carboxylic acid groups via a crosslinker; and
a methylene blue redox reporter conjugated to the single-stranded DNA aptamer.

2. The electrochemical aptasensor of claim 1, wherein the crosslinker is N-(3-Dimethylaminopropyl)-N-Ethylcarbodiimide.

3. The electrochemical aptasensor of claim 2, wherein the single-stranded DNA aptamer comprises a 5′-end having an amine group, wherein the amine group is conjugated to the carboxylic acid groups of the chemically oxidized onion-like carbon nanoparticles through the crosslinker.

4. The electrochemical aptasensor of claim 3, wherein the single-stranded DNA aptamer comprises a 3′-end having a thiol group, wherein the methylene blue redox reporter is conjugated to the thiol group via a thiol-maleimide Michael addition reaction.

5. The electrochemical aptasensor of claim 4, wherein the methylene blue redox reporter is methylene blue maleimide.

6. The electrochemical aptasensor of claim 1, wherein the working electrode is a glassy carbon electrode.

7. The electrochemical aptasensor of claim 1, wherein the electrochemical aptasensor is configured to exhibit a signal-off response upon binding of glial fibrillary acidic protein to the single-stranded DNA aptamer.

8. The electrochemical aptasensor of claim 7, wherein the electrochemical aptasensor is configured to detect glial fibrillary acidic protein in a concentration range from 0 pg/mL to 600 pg/mL.

9. The electrochemical aptasensor of claim 1, wherein the electrochemical aptasensor is configured to detect glial fibrillary acidic protein in a sweat sample.

10. The electrochemical aptasensor of claim 1, wherein the chemically oxidized onion-like carbon nanoparticles have diameters up to approximately 9.61 nm.

11. The electrochemical aptasensor of claim 1, wherein the single-stranded DNA aptamer is a GFAP-specific aptamer designated as Gli_233.

12. A method of fabricating a carbon-based ink for an electrochemical aptasensor, comprising:
preparing a suspension of chemically oxidized onion-like carbon nanoparticles in deionized water;
combining the suspension with a single-stranded DNA aptamer specific for glial fibrillary acidic protein and a crosslinker to form a first mixture;
incubating the first mixture to conjugate the single-stranded DNA aptamer to the chemically oxidized onion-like carbon nanoparticles through carboxylic acid groups on the chemically oxidized onion-like carbon nanoparticles;
adding a reducing agent to the first mixture to form a second mixture;
adding a methylene blue redox reporter to the second mixture to conjugate the methylene blue redox reporter to the single-stranded DNA aptamer; and
purifying the second mixture to remove unbound reagents and obtain the carbon-based ink.

13. The method of claim 12, wherein the suspension has a concentration of 0.02 mg/mL of chemically oxidized onion-like carbon nanoparticles.

14. The method of claim 12, wherein:
the crosslinker is N-(3-Dimethylaminopropyl)-N-Ethylcarbodiimide;
the reducing agent is Tris (2-Carboxyethyl) Phosphine Hydrochloride; and
the methylene blue redox reporter is methylene blue maleimide.

15. The method of claim 14, wherein incubating the first mixture comprises incubating overnight at room temperature, and wherein adding the reducing agent comprises allowing the second mixture to stand for 2 hours.

16. The method of claim 12, wherein purifying the second mixture comprises dialysis using dialysis cassettes followed by centrifugation using centrifugal filter units.

17. A method of detecting glial fibrillary acidic protein in a sample, comprising:
providing an electrochemical aptasensor comprising a working electrode having a carbon-based ink disposed thereon, the carbon-based ink comprising:
chemically oxidized onion-like carbon nanoparticles;
a single-stranded DNA aptamer specific for glial fibrillary acidic protein conjugated to the chemically oxidized onion-like carbon nanoparticles; and
a methylene blue redox reporter conjugated to the single-stranded DNA aptamer;
contacting the electrochemical aptasensor with the sample;
performing square wave voltammetry to measure an electrochemical signal from the methylene blue redox reporter; and
determining a concentration of glial fibrillary acidic protein in the sample based on a change in the electrochemical signal.

18. The method of claim 17, wherein the sample is a sweat sample.

19. The method of claim 17, wherein performing square wave voltammetry comprises scanning over a potential window from −0.5 V to 0.1 V versus Ag|AgCl at a frequency of 40 Hz, and wherein the electrochemical signal exhibits a peak at approximately −0.2 V versus Ag|AgCl.

20. The method of claim 19, wherein determining the concentration comprises correlating a decrease in peak current of the electrochemical signal with an increase in glial fibrillary acidic protein concentration according to a signal-off detection mechanism.
